(19) United States
(12) Patent Application Publication
(10) Pub. No.: US 2024/0254210 A1
(43) Pub. Date: Aug. 1, 2024

(54) BRAIN TARGETING COMPOSITIONS AND METHODS OF USE THEREOF

(71) Applicant: Genentech, Inc., South San Francisco, CA (US)

(72) Inventors: Michael John Dolton, South San Francisco, CA (US); Angelica Linnea Quartino, South San Francisco, CA (US); Kaycee Michelle Sink, South San Francisco, CA (US); Geoffrey Allen Kerchner, South San Francisco, CA (US); Aaron David Chesterman, South San Francisco, CA (US)

(21) Appl. No.: 18/417,787

(22) Filed: Jan. 19, 2024

Related U.S. Application Data
(63) Continuation of application No. PCT/US2022/074007, filed on Jul. 21, 2022.
(60) Provisional application No. 63/227,895, filed on Jul. 30, 2021.
(60) Provisional application No. 63/224,848, filed on Jul. 22, 2021.

(51) Int. Cl. C07K 16/18; A61K 9/00; A61K 39/00; A61K 47/42; A61P 25/28

(52) U.S. Cl. CPC C07K 16/18; A61K 9/0019; A61K 47/42; A61P 25/28; A61K 2039/505; A61K 2039/54; C07K 2317/24

(57) ABSTRACT

The present disclosure provides methods for treating Alzheimer's disease by subcutaneously administering a high volume and high dose of a brain targeting antibody or antigen-binding fragment thereof and provides compositions comprising an anti-amyloid β antibody or antigen-binding fragment thereof.

### CROSS-REFERENCE TO RELATED APPLICATIONS

This application is a continuation of International Application No. PCT/US2022/074007, filed Jul. 21, 2022, which claims priority to and benefit of U.S. Provisional Application No. 63/224,848, filed Jul. 22, 2021, and U.S. Provisional Application No. 63/227,895, filed Jul. 30, 2021, the contents of which are hereby incorporated by reference in their entireties.

### SEQUENCE LISTING

The instant application contains a Sequence Listing which has been submitted electronically in XML format and is hereby incorporated by reference in its entirety. Said XML copy, created on Jan. 19, 2024, is named 000218-0048-101_SL.xml and is 13,816 bytes in size.

### BACKGROUND

Traditional intravenous (IV) therapeutics can be associated with longer administration times, increased healthcare costs, and reduced convenience, compared with subcutaneous (SC) administration. IV therapies require administration by skilled healthcare professionals—usually in a hospital or clinic—thus limiting patient access to medications, and increasing healthcare resource burden. SC delivery can offer increased convenience to patients through the potential for self-administration and/or administration in a wider range of care settings beyond hospital-based clinics, while maintaining efficacy and safety compared with IV formulations of the same drug. However, the volume of fluid that can be administered subcutaneously is limited for large molecules, such as monoclonal antibodies or other viscous fluids, due to the restrictions of the extracellular matrix. Historically, tolerability issues such as infusion-site swelling have limited the use of higher dose/volume of SC administration (>10-15 mL), although reduced bioavailability is also a potential concern. These limitations mean that SC formulations may require multiple SC infusion or infusion sites, more frequent administration, and dose adjustment to achieve drug exposure equivalent to that achieved with an IV formulation. Systemic administration of therapeutics is also limiting due to the need of very high dosage levels of the therapeutic.

There remains a need for methods and compositions for administering high dose/high volumes of therapeutics, particularly for therapeutics that target the brain.

### SUMMARY

The present disclosure provides compositions suitable for subcutaneous administration comprising a high volume and a high dose of a brain targeting antibody or antigen-binding fragment thereof. These compositions are useful for treating, e.g., Alzheimer's disease.

In one aspect, the disclosure provides a method for treating Alzheimer's disease in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method for treating a subject at risk for Alzheimer's disease, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method for treating cognitive impairment in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method for reducing cognitive impairment in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL. In some embodiments, the cognitive impairment is mild cognitive impairment (MCI).

In one aspect, the disclosure provides a method of delaying progression of Alzheimer's Disease (AD) in a subject diagnosed with early or mild to moderate AD comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method of treating early or mild to moderate AD without increasing the risk of an adverse event comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method of delaying progression in a patient diagnosed with early or mild to moderate Alzheimer's Disease (AD) comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method of slowing clinical decline in a patient diagnosed with early or mild to moderate Alzheimer's Disease (AD) comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In some embodiments, the concentration of the brain targeting antibody or antigen-binding fragment thereof is about 140 mg/mL to about 190 mg/mL. In some embodiments, the concentration of the brain targeting antibody or antigen-binding fragment thereof is about 150 mg/mL to about 180 mg/mL. In some embodiments, the concentration of the brain targeting antibody or antigen-binding fragment thereof is about 150 mg/mL. In some embodiments, the concentration of the brain targeting antibody or antigen-binding fragment thereof is about 170 mg/mL. In some embodiments, the concentration of the brain targeting antibody or antigen-binding fragment thereof is about 180 mg/mL.

In some embodiments, the brain targeting antibody or antigen-binding fragment thereof is administered at a dose between about 400 mg and about 7500 mg. In some embodiments, the dose of the brain targeting antibody or antigen-binding fragment thereof is about 600 mg to about 7200 mg. In some embodiments, the dose of the brain targeting antibody or antigen-binding fragment thereof is about 600 mg. In some embodiments, the dose of the brain targeting antibody or antigen-binding fragment thereof is about 1200 mg. In some embodiments, the dose of the brain targeting antibody or antigen-binding fragment thereof is about 1700 mg. In some embodiments, the dose of the brain targeting antibody or antigen-binding fragment thereof is about 1800 mg. In some embodiments, the dose of the brain targeting antibody or antigen-binding fragment thereof is about 2400 mg. In some embodiments, the dose of the brain targeting antibody or antigen-binding fragment thereof is about 3400 mg. In some embodiments, the dose of the brain targeting antibody or antigen-binding fragment thereof is about 3600 mg. In some embodiments, the dose of the brain targeting antibody or antigen-binding fragment thereof is about 4320 mg. In some embodiments, the dose of the brain targeting antibody or antigen-binding fragment thereof is about 5760 mg. In some embodiments, the dose of the brain targeting antibody or antigen-binding fragment thereof is about 6800 mg. In some embodiments, the dose of the brain targeting antibody or antigen-binding fragment thereof is about 7200 mg.

In some embodiments, the brain targeting antibody or antigen-binding fragment thereof is administered with an infusion volume of about 4 mL to about 60 mL. In some embodiments, the infusion volume is about 10 mL to about 40 mL. In some embodiments, the infusion volume is about 4 mL. In some embodiments, the infusion volume is about 8 mL. In some embodiments, the infusion volume is about 10 mL. In some embodiments, the infusion volume is about 12 mL. In some embodiments, the infusion volume is about 16 mL. In some embodiments, the infusion volume is about 20 mL. In some embodiments, the infusion volume is about 24 mL. In some embodiments, the infusion volume is about 32 mL.

In some embodiments, the infusion volume is about 40 mL.

In some embodiments, the brain targeting antibody or antigen-binding fragment thereof is administered with a flow rate of about 1 mL/min to about 5 mL/min. In some embodiments, the flow rate is about 2 mL/min to about 4 mL/min. In some embodiments, the flow rate is about 2 mL/min. In some embodiments, the flow rate is about 4 mL/min.

In some embodiments, the method comprises further administering to the subject a permeation enhancer. In some embodiments, the permeation enhancer is hyaluronidase (e.g., Amphadase®, Hydase®, Hylenex® and Vitrase®). In some embodiments, the permeation enhancer is a recombinant human hyaluronidase. In some embodiments, the recombinant human hyaluronidase is a human soluble PH20 hyaluronidase glycoprotein, such as rHuPH20. In some embodiments, the brain targeting antibody or antigen-binding fragment thereof and the permeation enhancer are administered simultaneously. In some embodiments, the brain targeting antibody or antigen-binding fragment thereof and the permeation enhancer are administered consecutively. In some embodiments, the brain targeting antibody or antigen-binding fragment thereof and the permeation enhancer are in the same composition. In some embodiments, the brain targeting antibody or antigen-binding fragment thereof and the permeation enhancer are in separate compositions.

In some embodiments, the permeation enhancer is at a concentration of about 300 U/mL to about 2200 U/mL. In some embodiments, the concentration of the permeation enhancer is about 500 U/mL to about 2000 U/mL. In some embodiments, the concentration of the permeation enhancer is about 500 U/mL. In some embodiments, the concentration of the permeation enhancer is about 1000 U/mL. In some embodiments, the concentration of the permeation enhancer is about 2000 U/mL.

In some embodiments, the hyaluronidase is at a concentration of about 300 U/mL to about 2200 U/mL. In some embodiments, the concentration of the hyaluronidase is about 500 U/mL to about 2000 U/mL. In some embodiments, the concentration of the hyaluronidase is about 500 U/mL. In some embodiments, the concentration of the hyaluronidase is about 1000 U/mL. In some embodiments, the concentration of the hyaluronidase is about 2000 U/mL.

In some embodiments, the recombinant human hyaluronidase is at a concentration of about 300 U/mL to about 2200 U/mL. In some embodiments, the concentration of the recombinant human hyaluronidase is about 500 U/mL to about 2000 U/mL. In some embodiments, the concentration of the recombinant human hyaluronidase is about 500 U/mL. In some embodiments, the concentration of the recombinant human hyaluronidase is about 1000 U/mL. In some embodiments, the concentration of the recombinant human hyaluronidase is about 2000 U/mL.

In some embodiments, the composition is administered into a quadrant of the abdomen. In some embodiments, the composition is administered as one or more doses.

In some embodiments, the Alzheimer's disease is autosomal-dominant Alzheimer's disease. In some embodiments, the autosomal-dominant Alzheimer's disease is prodromal, mild, moderate, or mild-to-moderate. In some embodiments, the autosomal-dominant Alzheimer's disease is mild-to-moderate. In some embodiments, the Alzheimer's disease is sporadic AD. In some embodiments, the Alzheimer's disease is early or mild AD.

In some embodiments, the brain targeting antibody is a humanized monoclonal IgG4 antibody. In some embodiments, the brain targeting antibody is an anti-amyloid β antibody. In some embodiments, the anti-amyloid β antibody or antigen-binding fragment thereof comprises: (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6. In some embodiments, the anti-amyloid β antibody or antigen-binding fragment thereof comprises a VH domain comprising the amino acid sequence of SEQ ID NO: 7. In some embodiments, the anti-amyloid β antibody or antigen-binding fragment thereof comprises a VL domain comprising the amino acid sequence of SEQ ID NO: 8. In some embodiments, the anti-amyloid β antibody comprises a heavy chain comprising the amino acid sequence of SEQ ID NO: 9. In some embodiments, the anti-amyloid β antibody comprises a light chain comprising the amino acid sequence of SEQ ID NO: 10. In some embodiments, the anti-amyloid β antibody is crenezumab.

In some embodiments, the subject is a human.

In some embodiments, the disclosure provides a method for treating Alzheimer's disease in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and the human hyaluronidase (at a dose of about 2000 U/mL) are administered on Day 1 and the second dose and the hyaluronidase (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and hyaluronidase are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and the hyaluronidase (at a dose of about 2000 U/mL) are administered on Day 1 and the second dose and the hyaluronidase (at a dose of about 500 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating Alzheimer's disease in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the brain targeting antibody or antigen-binding fragment thereof is anti-amyloid β antibody or antigen-binding fragment thereof. In some embodiments, the anti-amyloid β antibody or antigen-binding fragment thereof comprises: (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6. In some embodiments, the anti-amyloid β antibody or antigen-binding fragment thereof comprises a VH domain comprising the amino acid sequence of SEQ ID NO: 7. In some embodiments, the anti-amyloid β antibody or antigen-binding fragment thereof comprises a VL domain comprising the amino acid sequence of SEQ ID NO: 8. In some embodiments, the anti-amyloid β antibody comprises a heavy chain comprising the amino acid sequence of SEQ ID NO: 9. In some embodiments, the anti-amyloid β antibody comprises a light chain comprising the amino acid sequence of SEQ ID NO: 10. In some embodiments, the anti-amyloid β antibody is crenezumab.

In some embodiments, the Alzheimer's disease is autosomal-dominant Alzheimer's disease. In some embodiments, the autosomal-dominant Alzheimer's disease is prodromal, mild, moderate, or mild-to-moderate. In some embodiments, the autosomal-dominant Alzheimer's disease is mild-to-moderate. In some embodiments, the Alzheimer's disease is sporadic AD. In some embodiments, the Alzheimer's disease is early or mild AD.

In one aspect the disclosure provides a composition suitable for administering a high volume and high dose of an anti-amyloid β antibody or antigen binding fragment thereof. In one aspect, the disclosure provides a composition comprising about 400 mg to about 7500 mg of an anti-amyloid β antibody or antigen binding fragment thereof. In one aspect, the disclosure provides a composition comprising about 600 mg to about 7200 mg of an anti-amyloid β antibody or antigen binding fragment thereof. In some embodiments, the composition comprises about 600 mg of the anti-amyloid β antibody or antigen binding fragment thereof. In some embodiments, the composition comprises about 1200 mg of the anti-amyloid β antibody or antigen binding fragment thereof. In some embodiments, the composition comprises about 1700 mg the anti-amyloid β antibody or antigen binding fragment thereof. In some embodiments, the composition about 1800 mg comprises the anti-amyloid β antibody or antigen binding fragment thereof. In some embodiments, the composition comprises about 2400 mg the anti-amyloid β antibody or antigen-binding fragment thereof. In some embodiments, the composition comprises about 3400 mg the anti-amyloid β antibody or antigen binding fragment thereof. In some embodiments, the composition comprises about 3600 mg the anti-amyloid β antibody or antigen binding fragment thereof. In some embodiments, the composition about 4320 mg comprises the anti-amyloid β antibody or antigen binding fragment thereof. In some embodiments, the composition comprises about 5760 mg the anti-amyloid β antibody or antigen binding fragment thereof. In some embodiments, the composition comprises about 6800 mg the anti-amyloid β antibody or antigen binding fragment thereof. In some embodiments, the composition comprises about 7200 mg the anti-amyloid β antibody or antigen binding fragment thereof.

In some embodiments, the composition further comprises a permeation enhancer. In some embodiments, the composition further comprises hyaluronidase (e.g., Amphadase®, Hydase®, Hylenex® and Vitrase®). In some embodiments, the composition further comprises recombinant human hyaluronidase. In some embodiments, the recombinant human hyaluronidase is a human soluble PH20 hyaluronidase glycoprotein, such as rHuPH20.

In some embodiments, the permeation enhancer is about 500 U/mL to about 2000 U/mL. In some embodiments, the concentration of the permeation enhancer is about 500 U/mL. In some embodiments, the concentration of permeation enhancer is about 1000 U/mL. In some embodiments, the concentration of the permeation enhancer is about 2000 U/mL.

In some embodiments, the hyaluronidase is about 500 U/mL to about 2000 U/mL. In some embodiments, the concentration of the hyaluronidase is about 500 U/mL. In some embodiments, the concentration of the hyaluronidase is about 1000 U/mL. In some embodiments, the concentration of the hyaluronidase is about 2000 U/mL.

In some embodiments, the recombinant human hyaluronidase is about 500 U/mL to about 2000 U/mL. In some embodiments, the concentration of the recombinant human hyaluronidase is about 500 U/mL. In some embodiments, the concentration of the recombinant human hyaluronidase is about 1000 U/mL. In some embodiments, the concentration of the recombinant human hyaluronidase is about 2000 U/mL.

In some embodiments, the disclosure provides a composition comprising about 130 mg/mL to about 200 mg/mL of an anti-amyloid β antibody or antigen-binding fragment thereof.

In some embodiments, the concentration of the anti-amyloid β antibody or antigen-binding fragment thereof is about 140 mg/mL to about 190 mg/mL. In some embodiments, the concentration of the anti-amyloid β antibody or antigen-binding fragment thereof is about 150 mg/mL to about 180 mg/mL. In some embodiments, the concentration of the anti-amyloid antibody or antigen-binding fragment thereof is about 150 mg/mL. In some embodiments, the concentration of the anti-amyloid β antibody or antigen-binding fragment thereof is about 170 mg/mL. In some embodiments, the concentration of the anti-amyloid β antibody or antigen-binding fragment thereof is about 180 mg/mL.

In some embodiments, the composition is suitable for administering the anti-amyloid β antibody or antigen-binding fragment thereof with an infusion volume of about 4 mL to about 60 mL. In some embodiments, the infusion volume is about 10 mL to about 40 mL. In some embodiments, the infusion volume is about 4 mL. In some embodiments, the infusion volume is about 8 mL. In some embodiments, the infusion volume is about 10 mL. In some embodiments, the infusion volume is about 12 mL. In some embodiments, the infusion volume is about 16 mL. In some embodiments, the infusion volume is about 20 mL. In some embodiments, the infusion volume is about 24 mL. In some embodiments, the infusion volume is about 32 mL. In some embodiments, the infusion volume is about 40 mL.

In some embodiments, the composition is suitable for administering the anti-amyloid β antibody or antigen-binding fragment thereof with a flow rate of about 1 mL/min to about 5 mL/min. In some embodiments, the flow rate is about 2 mL/min to about 4 mL/min. In some embodiments, the flow rate is about 2 mL/min. In some embodiments, the flow rate is about 4 mL/min.

In some embodiments, the anti-amyloid β antibody or antigen-binding fragment thereof comprises: (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6.

In some embodiments, the anti-amyloid β antibody or antigen-binding fragment thereof comprises a VH domain comprising the amino acid sequence of SEQ ID NO: 7.

In some embodiments, the anti-amyloid β antibody or antigen-binding fragment thereof comprises a VL domain comprising the amino acid sequence of SEQ ID NO: 8. In some embodiments, the anti-amyloid β antibody comprises a heavy chain comprising the amino acid sequence of SEQ ID NO: 9. In some embodiments, the anti-amyloid β antibody comprises a light chain comprising the amino acid sequence of SEQ ID NO: 10. In some embodiments, the anti-amyloid β antibody is crenezumab.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B. Study designs and treatment schema. FIG. 1A. Study 1 single ascending dose study. FIG. 1B. Study 2 multiple dose with or without recombinant human hyaluronidase (rHuPH20) study.

FIG. 2. Study 1 VAS pain scores following subcutaneous (SC) infusions (all cohorts). Dur represents during. SC represents subcutaneous. VAS represents visual analog scale. VAS is measured on a scale of 0 to 100 mm. Baseline, following catheter insertion but prior to infusion (pre-dose); 0 min dur, a retrospective assessment collected at time 0 minutes to describe how the subject felt while receiving the infusion; immediately after syringe pump stopped and prior to catheter removal; 0 min now, immediately after the infusion; immediately after syringe pump stopped and prior to catheter removal; t=5, 5 min post-infusion; t=20, 20 min post-infusion, t=60, 60 min post-infusion. The whiskers represent the highest and lowest non-outlier values, the box represents the upper and lower quartiles with the midline as the median. Outlier data are represented above the whiskers. An outlier represented an extreme value that differed greatly from other values in a set of values. An extreme value was considered to be an outlier if it was at least 1.5 interquartile ranges below the first quartile or at least 1.5 interquartile ranges above the third quartile. The dashed line represents the median.

FIGS. 3A-3F. Study 2 VAS pain scores following SC infusions for (FIG. 3A) Cohort 2, (FIG. 3B) Cohort 3, (FIG. 3C) Cohort 4, (FIG. 3D) Cohort 5 (Day 1), (FIG. 3E) Cohort 5A+5B and (FIG. 3F) Cohort 6 (Day 1+Day 15). VAS represents visual analog scale. VAS is measured on a scale of 0 to 100 mm. Baseline, following catheter insertion but prior to infusion (pre-dose). During infusion, a retrospective assessment collected at time 0 minutes to describe how the subject felt while receiving the infusion; immediately after syringe pump stopped and prior to catheter removal; t=0, immediately after the infusion; immediately after syringe pump stopped and prior to catheter removal; t=5, 5 min post-infusion; t=20, 20 min post-infusion, t=60, 60 min post-infusion.

FIG. 4. Area of infusion site erythema in Study 2, and cohorts E (20 mL crenezumab) and H (40 mL crenezumab) of Study 1 stratified by infusion volume and rHuPH20 co-administration at the 0 minute post-infusion (Panel A) and 60 minute post-infusion timepoint (Panel B). Solid line represents median, dashed lines represent the 25th and 75th percentiles. Cren represents crenezumab. rHuPH20 represents recombinant human hyaluronidase.

FIGS. 5A and 5B. Mean serum concentration-time profiles following crenezumab SC infusion in (a) Study 1 and (b) Study 2. Study 1: single ascending doses of SC crenezumab (600 mg to 7200 mg) for Cohorts A-H. Study 2: Cohort 2: Infusion 1, 1700 mg crenezumab (10 mL at 2 mL/min) (SC); Infusion 2, 3400 mg crenezumab (20 mL at 2 mL/min) (SC). Cohort 3: Infusion 1, 3400 mg crenezumab+2000 U/mL rHuPH20 (20 mL at 4 mL/min) (SC); Infusion 2, 6800 mg crenezumab+1000 U/mL rHuPH20 (40 mL at 4 mL/min) (SC). Cohort 4: Infusion 1, 1700 mg crenezumab (10 mL at 4 mL/min) (SC); Infusion 2, 3400 mg crenezumab (20 mL at 4 mL/min) (SC). Cohort 5A: Infusion 1, 3400 mg crenezumab (20 mL at 4 mL/min) (SC); Infusion 2, 6800 mg crenezumab+500 U/mL rHuPH20 (40 mL at 4 mL/min) (SC). Cohort 5B: Infusion 1, 3400 mg crenezumab+2000 U/mL rHuPH20 (20 mL at 4 mL/min) (SC); Infusion 2, 6800 mg crenezumab+500 U/mL rHuPH20 (40 mL at 4 mL/min) (SC). Profiles for Study 2 are for Day 15 dose in Cohorts 2-5, all of these participants received a dose on Day 1, which had not washed out by Day 15. rHuPH20 represents recombinant human hyaluronidase. SC represents subcutaneous.

FIG. 6. Study 1 boxplots of verbal descriptive scale (VDS) data for all participants. Dur represents during. VDS represents verbal descriptive scale. The scale used for VDS is: 1=Description No Pain, 2=Very Mild, 3=Mild, 4=Not Very Severe, 5=Quite Severe, 6=Very Severe 7=Almost Unbearable. The whiskers represent the highest and lowest nonoutlier values, the box represents the upper and lower quartiles with the midline as the median. Outlier data are represented above the whiskers. An outlier represented an extreme value that differed greatly from other values in a set of values. An extreme value was considered to be an outlier if it was at least 1.5 interquartile ranges below the first quartile or at least 1.5 interquartile ranges above the third quartile. The dashed line represents the median.

FIGS. 7A-7F. Study 2 boxplots of verbal descriptive scale (VDS) data for (FIG. 7A) Cohort 2, (FIG. 7B) Cohort 3, (FIG. 7C) Cohort 4, (FIG. 7D) Cohort 5 (Day 1), (FIG. 7E) Cohort 5A+5B and (FIG. 7F) Cohort 6 (Day 1+Day 15). Dur represents during. VDS represents verbal descriptive scale. The scale used for VDS is: 1=Description No Pain, 2=Very Mild, 3=Mild, 4=Not Very Severe, 5=Quite Severe, 6=Very Severe 7=Almost Unbearable. VDS was only assessed on the baseline and t=0 timepoints.

FIG. 8. Distribution of tissue back pressure following placebo injections (Cohort 6, Study 2). rHuPH20 represents recombinant human hyaluronidase. Tissue back pressure is measured in psi.

FIGS. 9A.I, 9A.II, 9B.I, and 9B.II. Visual predictive check (VPC) of the final model. FIGS. 9A.I and 9A.II) Prediction corrected visual prediction check of the crenezumab population PK model stratified by study and route of administration in linear scale. FIGS. 9B.I and 9B.II) Prediction corrected visual prediction check of the crenezumab population PK model stratified by study and route of administration in log scale. IV represents intravenous. PK represents pharmacokinetic. SC represents subcutaneous. Open circles are observed crenezumab concentrations displayed versus time; lines represent the median, 5th and 95th percentiles of the observations; the shaded red and blue areas represent the 95% confidence interval of the median, 5th and 95th percentiles predicted by the model.

FIG. 10. Crenezumab SC bioavailability individual estimates from the population PK model by study, dose (Study 1) or cohort (Study 2). Line at median. PK represents pharmacokinetic. rHuPH20 represents recombinant human hyaluronidase. SC represents subcutaneous.

FIGS. 11A-11C. Demographic and baseline characteristics of Study 1 (single ascending dose study) and Study 2 (multiple dose—rHuPH20 study). BMI represents body mass index. rHuPH20 represents recombinant human hyaluronidase. SC represents subcutaneous. SD represents standard deviation.

FIGS. 12A-12C. Summary table of Local Injection Site Symptom Assessment (LISSA) of Study 1 (single ascending dose study) and Study 2 (multiple dose—rHuPH20 study). C represents crenezumab injection. Inj represents injection. IV represents intravenous. P represents placebo injection. R represents reference placebo injection. rHuPH20 represents recombinant human hyaluronidase. SC represents subcutaneous. T represents test placebo injection. ^{a} represents most common ‘Other’ injection site reactions were induration and swelling. Tenderness, raised red area/raised redness, edema, and pain were also reported more than once but to a far lesser extent. All other reactions (including sensitivity, papules, stinging, wheal formation, and blister fluid filled intact) were reported only 1 time each. ^{b} represents not all participants assessable for these reactions at certain timepoints. Data represent the sum of injection site reaction events captured at different timepoints post-injection.

FIGS. 13A-13C. Study table of treatment-emergent adverse events of Study 1 (single ascending dose study) and Study 2 (multiple dose—rHuPH20 study). AE represents adverse event. IV represents intravenous. rHuPH20 represents recombinant human hyaluronidase. SAE represents serious adverse event. SC represents subcutaneous. ^{a} represents Injection one only: participants in Cohort 5A received crenezumab alone and participants in Cohort 5B received crenezumab+rHuPH20. ^{b} represents Injection two only: all participants received crenezumab+rHuPH20.

FIG. 14. Table of parameter estimates from the crenezumab population PK model. ^{a} represents SD of logistic distribution. CV represents coefficient of variation. IIV represents interindividual variability. RSE represents relative standard error. SHR represents shrinkage.

### DETAILED DESCRIPTION

The present disclosure provides compositions suitable for subcutaneous administration comprising a high volume and a high dose of a brain targeting antibody or antigen-binding fragment thereof and methods of use thereof for treating cognitive impairment including, e.g., Alzheimer's disease.

### Definitions

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Fourth edition, Cold Spring Harbor Laboratory Press, 2014; Ausubel et al., CURRENT PROTOCOLS IN MOLECULAR BIOLOGY, John Wiley & Sons, New York, 2012; the series METHODS IN ENZYMOLOGY, Academic Press, San Diego; Wolffe, Murphy et al. JANEWAY'S IMMUNOBIOLOGY, Tenth Edition, W. W. Norton & Company, 2022.

The term “herein” means the entire application.

Unless otherwise defined herein, scientific and technical terms used in this application shall have the meanings that are commonly understood by those of ordinary skill in the art to which this disclosure belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the multiple dependent claims.

Any publications, patents and published patent applications referred to in this application are specifically incorporated by reference herein. In case of conflict, the present specification, including its specific definitions, will control.

Throughout this specification, the word “comprise” or variations such as “comprises” or “comprising,” which is synonymous with “including,” “containing,” or “characterized by,” is inclusive or open-ended and does not exclude additional, unrecited elements or method steps.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present application. The materials, methods, and examples are illustrative only and not intended to be limiting.

It should be understood that any of the embodiments described herein, including those described under different aspects of the disclosure and different parts of the specification (including embodiments described only in the Examples) can be combined with one or more other embodiments of this disclosure, unless explicitly disclaimed or improper. Combination of embodiments are not limited to those specific combinations claimed via the multiple dependent claims.

“Administering” or “administration of” a substance, a compound or an agent to a subject refers to the contact of that substance, compound or agent to the subject or a cell, tissue, organ or bodily fluid of the subject. Such administration can be carried out using one of a variety of methods known to those skilled in the art. For example, a compound or an agent can be administered parenterally, such as subcutaneously. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a subject to self-administer a drug, or to have the drug administered by another and/or who provides a subject with a prescription for a drug is administering the drug to the subject.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, full-length or intact antibodies, etc.) that specifically bind to a given antigen. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. There are two types of light chain: λ and κ. In humans, λ and κ light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety).

The terms “subject” and “patient” are used interchangeably herein and refer to mammals including, but not limited to, human and non-human animals. These terms include mammals, such as humans, and primates (e.g., monkey). In some embodiments, the subject is a human. Accordingly, the term “subject” or “patient” as used herein means any mammalian patient or subject to which the compositions of the disclosure may be administered.

The term “treatment” (and grammatical variations thereof such as “treat” or “treating”) refers to clinical intervention in an attempt to alter the natural course of the individual being treated, and can be performed during the course of clinical pathology. Desirable effects of treatment include, but are not limited to, alleviation or amelioration of one or more symptoms, diminishment of or delay in the appearance of or worsening of any direct or indirect pathological consequences of the disease, decrease of the rate of disease progression, and amelioration or palliation of the disease state. In some embodiments, antibodies are used to delay development of a disease or to slow the progression of a disease.

The terms “therapeutically effective amount” and “effective amount” are used interchangeably herein and refer to that amount of the therapeutic agent being administered, as a single agent or in combination with one or more additional agents, which will relieve to some extent one or more of the symptoms of the condition being treated. In some embodiments, the therapeutically effective amount is an amount sufficient to effect the beneficial or desired clinical results. With respect to the treatment of a condition (e.g., Alzheimer's disease), a therapeutically effective amount refers to that amount which has at least one of the following effects: palliate, ameliorate, stabilize, reverse, prevent, slow or delay the progression of (and/or symptoms associated with) of the condition, such as to modify the progression of AD, particularly mild-to-moderate AD, and/or to alleviate and/or prevent one or more symptoms of AD. In some embodiments, an effective amount is used to reduce the rate of memory decline. The effective amounts that may be used in the present disclosure varies depending upon the manner of administration, the age, body weight, and general health of the subject. The appropriate amount and dosage regimen can be determined using routine skill in the art.

The term “therapeutic agent” refers to any agent that is used to treat a disease, including but not limited to an agent that treats a symptom of the disease.

As used herein, “delaying” or “slowing” the progression of a disease refers to preventing, deferring, hindering, slowing, retarding, stabilizing, and/or postponing development of the disease, such as to modify the progression of AD, particularly mild-to-moderate AD. This delay can be of varying lengths of time, depending on the history of the disease and/or individual being treated.

A “symptom,” as used herein, refers to a phenomenon or feeling of departure from normal function, sensation, or structure that is experienced by a subject.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al, Proc. Natl. Acad. Sci. USA 81:6851-6855 (1984)).

The “class” of an antibody refers to the type of constant domain or constant region possessed by its heavy chain. There are five major classes of antibodies: IgA, IgD, IgE, IgG, and IgM, and several of these may be further divided into subclasses (or “isotypes”), e.g., IgG1, IgG2, IgG3, IgG4, IgA1, and IgA2. The heavy chain constant domains that correspond to the different classes of immunoglobulins are called α, δ, ε, γ, and μ, respectively.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al, Nature 321:522-525 (1986); Riechmann et al, Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23: 1035-1038 (1995); Hurle and Gross, Curr. Op. Biotech. 5:428-433 (1994).

A “human antibody” is one which comprises an amino acid sequence corresponding to that of an antibody produced by a human or a human cell and/or has been derived from a non-human source that utilizes human antibody repertoires or other human antibody-encoding sequences, for example made using any of the techniques for making human antibodies as disclosed herein. Such techniques include, but are not limited to, screening human-derived combinatorial libraries, such as phage display libraries (see, e.g., Marks et al, J. Mol. Biol, 222: 581-597 (1991) and Hoogenboom et al, Nucl Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al, Monoclonal Antibody Production Techniques and Applications, pp. 55-93 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al, Proc. Natl. Acad. Sci USA, 90: 2551 (1993); Jakobovits et al., Nature, 362: 255 (1993); Bruggermann et al., Year in Immunol, 7: 33 (1993)). This definition of a human antibody specifically excludes a humanized antibody comprising antigen-binding residues from a non-human animal.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al, J. Chromatogr. B 848:79-87 (2007).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al, J. Immunol. 150:880-887 (1993); Clarkson et al, Nature 352:624-628 (1991).

The term “hypervariable region,” “HVR,” or “HV,” when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et ah, Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these HVRs are noted below in Table 1.

Hypervariable regions may comprise “extended hypervariable regions” as follows: 24-36 or 24-34 (LI), 46-56 or 49-56 or 50-56 or 52-56 (L2) and 89-97 (L3) in the VL and 26-35 (HI), 50-65 or 49-65 (H2) and 93-102, 94-102 or 95-102 (H3) in the VH. The variable domain residues are numbered according to Kabat et al, supra for each of these definitions.

TABLE 1

| Loop | Kabat | AbM | Chothia | Contact |
|---|---|---|---|---|
| LI | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| HI | H31-H35B | H26-H35B | H26-H32 | H30-H35B |
| | | | | (Kabat Numbering) |
| HI | H31-H35 | H26-H35 | H26-H32 | H30-H35 |
| | | | | (Chothia Numbering |
| H2 | H50-H65 | H50-H58 | H53-H55 | H47-H58 |
| H3 | H95-H102 | H95-H102 | H96-H101 | H93-H101 |

“Framework” or “FR” residues are those variable domain residues other than the hypervariable region residues as herein defined. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” or “binding affinity” refers to the strength of the sum total of noncovalent interactions between a single binding site of a molecule (e.g., an antibody) an its binding partner (e.g., antigen). Unless indicated otherwise, as used herein, “binding affinity” refers to intrinsic binding affinity which reflects a 1:1 interaction between members of a binding pair (e.g., antibody and antigen binding arm). The affinity of a molecule X for its partner Y can generally be represented by the dissociation constant (Kd). Affinity can be measured by common methods known in the art, including those described herein, any of which can be used for purposes of this disclosure. Specific illustrative and exemplary embodiments for measuring binding affinity are described herein.

An “affinity matured” antibody refers to an antibody with one or more alterations in one or more hypervariable regions (HVRs), compared to a parent antibody which does not possess such alterations, such alterations resulting in an improvement in the affinity of the antibody for antigen for antigen.

The terms “Anti-amyloid β,” “anti-Aβ,” “anti-Abeta,” “Anti-amyloid β immunoglobulin,” and “antibody that binds Aβ” are used interchangeably herein, and refer to an antibody that specifically binds to human Abeta (Aβ). A nonlimiting example of an anti-amyloid β antibody is crenezumab. Other non-limiting examples of anti-amyloid β antibodies are solanezumab, bapinezumab, aducanumab, and gantenerumab. In some embodiments, the anti-amyloid β antibody comprises: (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6. In some embodiments, the anti-amyloid β antibody comprises a VH domain comprising the amino acid sequence of SEQ ID NO: 7. In some embodiments, the anti-amyloid β antibody comprises a VL domain comprising the amino acid sequence of SEQ ID NO: 8. In some embodiments, the anti-amyloid β antibody comprises a heavy chain comprising the amino acid sequence of SEQ ID NO: 9. In some embodiments, the anti-amyloid β antibody comprises a light chain comprising the amino acid sequence of SEQ ID NO: 10. In some embodiments, the anti-amyloid β antibody is an IgG antibody. In some embodiments, the anti-amyloid β antibody is an IgG4 antibody. In some embodiments, the IgG4 antibody comprises a mutation in its constant domain such that serine 228 is instead a proline.

The terms “crenezumab” and “MABT5102A” are used interchangeably herein, and refer to a specific anti-amyloid β antibody that binds to monomeric, oligomeric, and fibril forms of Aβ, and which is associated with CAS registry number 1095207. Crenezumab comprises a heavy chain comprising the amino acid sequence of SEQ ID NO: 9 and a light chain comprising the amino acid sequence of SEQ ID NO: 10.

The terms “amyloid β,” “Aβ,” or “β-amyloid” are used interchangeably herein, are art recognized terms and refer to amyloid β proteins and peptides, amyloid β precursor protein (APP) (including that produced by β-secretase 1 cleavage), as well as modifications, fragments and any functional equivalents thereof. Amyloid β as used herein is meant any fragment produced by proteolytic cleavage of APP, including, but not limited to, those fragments which are involved in or associated with the amyloid pathologies including, but not limited to, Aβ₁₋₃₈, Aβ₁₋₃₉, Aβ₁₋₄₀, Aβ₁₋₄₁, Aβ₁₋₄₂, Aβ₁₋₄₃.

The structure and sequences of the amyloid β peptides as disclosed herein are well-known to those skilled in the art and methods of producing said peptides or of extracting them from brain and other tissues are described, for example, in Glenner and Wong, Biochem Biophys Res Comm 129, 885-890 (1984). Moreover, amyloid β peptides are also commercially available in various forms.

The term “specifically binds” in reference to an antibody refers to an antibody binding to its target antigen with greater affinity than it does to structurally different antigen(s).

A “human consensus framework” is a framework which represents the most commonly occurring amino acid residue in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. et al. et al.

The term “early Alzheimer's Disease” or “early AD” as used herein (e.g., a “patient diagnosed with early AD” or a “patient suffering from early AD”) includes patients with mild cognitive impairment, such as a memory deficit, due to AD and patients having AD biomarkers, for example amyloid positive patients.

The term “mild Alzheimer's Disease” or “mild AD” as used herein (e.g., a “patient diagnosed with mild AD”) refers to a stage of AD characterized by a Mini-Mental State Exam (MMSE) score of 20 to 26.

The term “mild to moderate Alzheimer's Disease” or “mild to moderate AD” as used herein encompasses both mild and moderate AD, and is characterized by an MMSE score of 18 to 26.

The term “moderate Alzheimer's Disease” or “moderate AD” as used herein (e.g., a “patient diagnosed with moderate AD”) refers to a stage of AD characterized by an MMSE score of 18 to 19.

The term “Amyloid-Related Imaging Abnormality (ARIA)” as used herein refers to a treatment emergent adverse event. In some embodiments, the ARIA is Amyloid-Related Imaging Abnormality—Edema. In some embodiments, the ARIA is Amyloid-Related Imaging Abnormality—Hemorrhage.

The term “Amyloid-Related Imaging Abnormality—Edema” or “ARIA-E” encompasses cerebral vasogenic edema and sulcal effusion. In some embodiments, the method of treating Alzheimer's disease of this disclosure does not increase the risk of a treatment emergent adverse event, wherein the adverse event is Amyloid-Related Imaging Abnormality—Edema (ARIA-E).

The term “Amyloid-Related Imaging Abnormality—Hemorrhage” or “ARIA-H” encompasses microhemorrhage and superficial siderosis of the central nervous system. In some embodiments, the method of treating Alzheimer's disease of this disclosure does not increase the risk of a treatment emergent adverse event, wherein the adverse event is Amyloid-Related Imaging Abnormality—Edema (ARIA-E).

The term “cerebral vasogenic edema” as used herein refers to an excess accumulation of intravascular fluid or protein in the intracellular or extracellular spaces of the brain. Cerebral vasogenic edema is detectable by, e.g., brain MRI, including, but not limited to FLAIR MRI, and can be asymptomatic (“asymptomatic vasogenic edema”) or associated with neurological symptoms, such as confusion, dizziness, vomiting, and lethargy (“symptomatic vasogenic edema”) (see Sperling et al. Alzheimer's & Dementia, 7:367, 2011).

The term “sulcal effusion” as used herein refers to effusion of fluid in the furrows, or sulci, of the brain. Sulcal effusions are detectable by, e.g., brain MRI, including but not limited to FLAIR MRI. See Sperling et al. Alzheimer's & Dementia, 7:367, 2011.

The term “superficial siderosis of the central nervous system” as used herein refers to bleeding or hemorrhage into the subarachnoid space of the brain and is detectable by, e.g., brain MRI, including but not limited to T2*-weighted GRE MRI. Symptoms indicative of superficial siderosis of the central nervous system include sensorineural deafness, cerebellar ataxia, and pyramidal signs. See Kumara-N, Am J Neuroradiol. 31:5, 2010.

The term “progression” as used herein refers to the worsening of a disease over time. The “progression rate” or “rate of progression” of a disease refers to how fast or slow a disease develops over time in a patient diagnosed with the disease. The progression rate of a disease can be represented by measurable changes over time of particular characteristics of the disease. A patient carrying particular genetic trait is said to have, or more likely to have, “increased progression rate” if her disease state progresses faster than those patients without such genetic trait. On the other hand, a patient responding to a therapy is said to have, or more likely to have, “decreased progression rate” if her disease progression slows down after the therapy, when compared to her disease state prior to the treatment or to other patients without the treatment.

The term “effector function” as used herein refers to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation. It is known in the art that wild-type IgG4 antibodies have less effector function than wild-type IgG1 antibodies.

The term “Fc region” as used herein refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

The terms “full length antibody,” “intact antibody,” and “whole antibody” are used herein interchangeably to refer to an antibody having a structure substantially similar to a native antibody structure or having heavy chains that contain an Fc region as defined herein.

The term “native antibody” or “native antibodies” as used herein refers to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The present disclosure provides compositions suitable for subcutaneous administration comprising a high volume and a high dose of a brain targeting antibody or antigen-binding fragment thereof. These compositions are useful for treating, e.g., Alzheimer's disease.

### Methods of Use

In one aspect, the disclosure provides a method for treating Alzheimer's disease in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method for treating a subject at risk for Alzheimer's disease, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method for treating cognitive impairment in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/ml to about 200 mg/mL. In some embodiments, the cognitive impairment is mild cognitive impairment (MCI). In some embodiments, the subject is suffering from Alzheimer's Disease.

In one aspect, the disclosure provides a method for reducing cognitive impairment in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL. In some embodiments, the cognitive impairment is mild cognitive impairment (MCI). In some embodiments, the subject is suffering from Alzheimer's Disease.

In one aspect, the disclosure provides a method of delaying progression of cognitive impairment in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL. In some embodiments, the cognitive impairment is mild cognitive impairment (MCI). In some embodiments, the subject is suffering from Alzheimer's Disease.

In one aspect, the disclosure provides a method of delaying progression of Alzheimer's Disease (AD) in a subject diagnosed with early or mild to moderate AD comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method of treating early or mild to moderate AD without increasing the risk of an adverse event comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In one aspect, the disclosure provides a method of slowing clinical decline in a patient diagnosed with early or mild to moderate Alzheimer's Disease (AD) comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

In some embodiments, the brain targeting antibody or antigen-binding fragment thereof is administered at a dose between about 400 mg and about 7500 mg. In some embodiments, the dose of the brain targeting antibody or antigen-binding fragment thereof is about 600 mg to about 7200 mg. In some embodiments, the brain targeting antibody or antigen-binding fragment thereof is administered at a dose of about 1700 mg, about 3400 mg or about 6800 mg. In some embodiments, the brain targeting antibody is administered at a dose between about 400 mg and about 7500 mg. In some embodiments, the dose of the brain targeting antibody is about 600 mg to about 7200 mg. In some embodiments, the brain targeting antibody is administered at a dose of about 1700 mg, about 3400 mg or about 6800 mg

In some embodiments, the brain targeting antibody or antigen-binding fragment thereof is administered with an infusion volume of about 4 mL to about 60 mL. In some embodiments, the infusion volume is about 10 mL to about 40 mL.

In some embodiments, the brain targeting antibody or antigen-binding fragment thereof is administered with a flow rate of about 1 mL/min to about 5 mL/min. In some embodiments, the flow rate is about 2 mL/min to about 4 mL/min.

In some embodiments, the method comprises further administering to the subject a permeation enhancer. In some embodiments, the composition further comprises hyaluronidase (e.g., Amphadase®, Hydase®, Hylenex® and Vitrase®). In some embodiments, the permeation enhancer is a recombinant human hyaluronidase. In some embodiments, the recombinant human hyaluronidase is a human soluble PH20 hyaluronidase glycoprotein, such as rHuPH20. In some embodiments, the permeation enhancer (e.g. hyaluronidase) is administered a dose of about 500 U/ml to about 2000 U/mL. In some embodiments, the permeation enhancer (e.g. hyaluronidase) is administered a dose of about 500 U/ml. In some embodiments, the permeation enhancer (e.g. hyaluronidase) is administered a dose of about 1000 U/ml. In some embodiments, the permeation enhancer (e.g. hyaluronidase) is administered a dose of about 1500 U/ml. In some embodiments, the permeation enhancer (e.g. hyaluronidase) is administered a dose of about 2000 U/ml.

In some embodiments, the brain targeting antibody or antigen-binding fragment thereof and the permeation enhancer are administered simultaneously. In some embodiments, the brain targeting antibody or antigen-binding fragment thereof and the permeation enhancer are administered consecutively. In some embodiments, the brain targeting antibody or antigen-binding fragment thereof and the permeation enhancer are in the same composition. In some embodiments, the brain targeting antibody or antigen-binding fragment thereof and the permeation enhancer are in separate compositions. In some embodiments, the anti-amyloid β antibody or antigen-binding fragment thereof and the hyaluronidase are administered simultaneously. In some embodiments, the anti-amyloid β antibody or antigen-binding fragment thereof and the hyaluronidase are administered consecutively. In some embodiments, the anti-amyloid β antibody or antigen-binding fragment thereof and the hyaluronidase are in the same composition. In some embodiments, the anti-amyloid β antibody or antigen-binding fragment thereof and the hyaluronidase are in separate compositions.

In some embodiments, the Alzheimer's disease is autosomal-dominant Alzheimer's disease. In some embodiments, the autosomal-dominant Alzheimer's disease is prodromal, mild, moderate, or mild-to-moderate. In some embodiments, the autosomal-dominant Alzheimer's disease is mild-to-moderate. In some embodiments, the Alzheimer's disease is sporadic AD. In some embodiments, the Alzheimer's disease is early or mild AD.

In some embodiments, the subject is a human.

In some embodiments, the method comprising subcutaneously administering to the subject a first dose of the brain targeting antibody (e.g. crenezumab) or an antigen-binding fragment thereof and second dose of the brain targeting antibody (e.g. crenezumab) or an antigen-binding fragment thereof. In some embodiments, the second dose comprises twice the amount of the brain targeting antibody (e.g. crenezumab) or antigen-binding fragment thereof as the first dose. In some embodiments, the first and second dose of the brain targeting antibody (e.g. crenezumab) or an antigen-binding fragment thereof are administered two weeks apart. In some embodiments, the first dose of the brain targeting antibody (e.g. crenezumab) or an antigen-binding fragment thereof is administered on Day 1 and the second dose of the brain targeting antibody (e.g. crenezumab) or an antigen-binding fragment thereof is administered on Day 15. In some embodiments, the first dose of the brain targeting antibody (e.g. crenezumab) or an antigen-binding fragment thereof is administered with a first dose of a permeation enhancer (e.g., hyaluronidase), and the second dose of the brain targeting antibody (e.g. crenezumab) or an antigen-binding fragment thereof is administered with a second dose of a permeation enhancer (e.g., hyaluronidase). In some embodiments, the second dose of the permeation enhancer (e.g. hyaluronidase) comprises half the amount of the permeation enhancer (e.g. hyaluronidase) as the first dose of the permeation enhancer (e.g. hyaluronidase). In some embodiments, the second dose of the permeation enhancer (e.g. hyaluronidase) comprises one quarter the amount of the permeation enhancer (e.g. hyaluronidase) as the first dose of the permeation enhancer (e.g. hyaluronidase).

In some embodiments, the disclosure provides a method for treating cognitive impairment in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for treating cognitive impairment in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and recombinant human hyaluronidase (at a dose of about 2000 U/mL) are administered on Day 1 and the second dose and permeation enhancer (at a dose of about 500 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for treating cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method for treating cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method for treating Alzheimer's disease in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with permeation enhancer at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and permeation enhancer are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating Alzheimer's disease in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for treating Alzheimer's disease in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method for treating Alzheimer's disease in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method for treating a subject at risk for Alzheimer's disease, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating a subject at risk for Alzheimer's disease, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for treating a subject at risk for Alzheimer's disease, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method for treating a subject at risk for Alzheimer's disease, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method for reducing cognitive impairment in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for reducing cognitive impairment in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for reducing cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for reducing cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method for reducing cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of delaying progression of cognitive impairment in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer permeation enhancer (at a dose of about 2000 U/mL) are administered on Day 1 and the second dose and permeation enhancer (at a dose of about 500 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of delaying progression of cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of delaying progression of cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method of delaying progression of cognitive impairment in a subject, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein:
(a) the first dose comprises about 3400 mg of a brain targeting antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein:
(a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein:
(a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered hyaluronidase at a dose of about 500 U/mL. In some embodiments, the first dose is administered on Day 1 and the second dose and the hyaluronidase are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of delaying progression in a subject diagnosed with early or mild to moderate Alzheimer's Disease (AD), the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of delaying progression in a subject diagnosed with early or mild to moderate Alzheimer's Disease (AD), the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method of delaying progression in a subject diagnosed with early or mild to moderate Alzheimer's Disease (AD), the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of treating early or mild to moderate AD without increasing the risk of an adverse event, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and
(b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and
(b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and
(b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and
(b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of treating early or mild to moderate AD without increasing the risk of an adverse event, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of treating early or mild to moderate AD without increasing the risk of an adverse event, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method of treating early or mild to moderate AD without increasing the risk of an adverse event, the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 2 mL/min; and (b) the second dose comprises about 3400 mg of a brain targeting antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein:
(a) the first dose comprises about 3400 mg of a brain targeting antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer e (at a dose of about 1000 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein:
(a) the first dose comprises about 3400 mg of a brain targeting antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein:
(a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 1700 mg of a brain targeting antibody or antigen-binding fragment thereof, and is administered in an infusion volume of 10 mL, and with a flow rate of about 4 mL/min; and (b) the second dose comprises about 3400 mg of a brain targeting antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL, and with a flow rate of about 4 mL/min; and (b) the second dose comprises about 6800 mg of a brain targeting antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of a brain targeting antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of slowing clinical decline in a patient diagnosed with early or mild to moderate Alzheimer's Disease (AD), the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL hyaluronidase.

In some embodiments, the disclosure provides a method of slowing clinical decline in a patient diagnosed with early or mild to moderate Alzheimer's Disease (AD), the method comprising subcutaneously administering to the subject a composition comprising about 6800 mg of a brain targeting antibody or antigen-binding fragment thereof, administered in an infusion volume of 40 mL and with a flow rate of about 4 mL/min; and a dose of about 500 U/mL recombinant human hyaluronidase.

In any of the foregoing embodiments, the brain targeting antibody or antigen-binding fragment thereof may be a full-length brain targeting antibody.

In some embodiments, the brain targeting antibody or antigen-binding fragment thereof is anti-amyloid β antibody or antigen-binding fragment thereof. In some embodiments, the anti-amyloid β antibody or antigen-binding fragment thereof comprises: (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6. In some embodiments, the anti-amyloid β antibody or antigen-binding fragment thereof comprises a VH domain comprising the amino acid sequence of SEQ ID NO: 7. In some embodiments, the anti-amyloid β antibody or antigen-binding fragment thereof comprises a VL domain comprising the amino acid sequence of SEQ ID NO: 8. In some embodiments, the anti-amyloid β antibody or antigen-binding fragment thereof comprises a VH domain comprising the amino acid sequence of SEQ ID NO: 7 and a VL domain comprising the amino acid sequence of SEQ ID NO: 8. In some embodiments, the anti-amyloid β antibody comprises a heavy chain comprising the amino acid sequence of SEQ ID NO: 9. In some embodiments, the anti-amyloid β antibody comprises a light chain comprising the amino acid sequence of SEQ ID NO: 10. In some embodiments, the anti-amyloid β antibody comprises a heavy chain comprising the amino acid sequence of SEQ ID NO: 9 and a light chain comprising the amino acid sequence of SEQ ID NO: 10. In some embodiments, the anti-amyloid β antibody is crenezumab.

In some embodiments, the anti-amyloid β antibody or antigen-binding fragment thereof is administered at a dose between about 400 mg and about 7500 mg. In some embodiments, the dose of the anti-amyloid β antibody or antigen-binding fragment thereof is about 600 mg to about 7200 mg. In some embodiments, the anti-amyloid β antibody or antigen-binding fragment thereof is administered at a dose of about 1700 mg, about 3400 mg or about 6800 mg. In some embodiments, the anti-amyloid β antibody is administered at a dose between about 400 mg and about 7500 mg. In some embodiments, the dose of the anti-amyloid β antibody is about 600 mg to about 7200 mg. In some embodiments, the anti-amyloid β antibody is administered at a dose of about 1700 mg, about 3400 mg or about 6800 mg. In some embodiments, crenezumab is administered at a dose between about 400 mg and about 7500 mg. In some embodiments, the dose of crenezumab is about 600 mg to about 7200 mg. In some embodiments, crenezumab is administered at a dose of about 1700 mg, about 3400 mg or about 6800 mg.

In some embodiments, the disclosure provides a method for treating cognitive impairment in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for treating cognitive impairment in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and recombinant human hyaluronidase (at a dose of about 2000 U/mL) are administered on Day 1 and the second dose and permeation enhancer (at a dose of about 500 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for treating cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method for treating cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method for treating Alzheimer's disease in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with permeation enhancer at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and permeation enhancer are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method for treating Alzheimer's disease in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating Alzheimer's disease in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for treating Alzheimer's disease in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method for treating Alzheimer's disease in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method for treating a subject at risk for Alzheimer's disease, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 1700 mg of an anti-amyloid antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method for treating a subject at risk for Alzheimer's disease, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for treating a subject at risk for Alzheimer's disease, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for treating a subject at risk for Alzheimer's disease, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method for treating a subject at risk for Alzheimer's disease, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method for reducing cognitive impairment in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method for reducing cognitive impairment in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method for reducing cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method for reducing cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method for reducing cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method for reducing cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of delaying progression of cognitive impairment in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer permeation enhancer (at a dose of about 2000 U/mL) are administered on Day 1 and the second dose and permeation enhancer (at a dose of about 500 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of delaying progression of cognitive impairment in a subject, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of delaying progression of cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of delaying progression of cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method of delaying progression of cognitive impairment in a subject, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein:
(a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein:
(a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein:
(a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered hyaluronidase at a dose of about 500 U/mL. In some embodiments, the first dose is administered on Day 1 and the second dose and the hyaluronidase are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of delaying progression in a subject diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of delaying progression in a subject diagnosed with early or mild to moderate Alzheimer's Disease (AD), the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of delaying progression in a subject diagnosed with early or mild to moderate Alzheimer's Disease (AD), the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method of delaying progression in a subject diagnosed with early or mild to moderate Alzheimer's Disease (AD), the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of treating early or mild to moderate AD without increasing the risk of an adverse event, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer (at a dose of about 1000 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of treating early or mild to moderate AD without increasing the risk of an adverse event, the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of treating early or mild to moderate AD without increasing the risk of an adverse event, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of treating early or mild to moderate AD without increasing the risk of an adverse event, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method of treating early or mild to moderate AD without increasing the risk of an adverse event, the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 2 mL/min; and (b) the second dose comprises about 3400 mg of an anti-amyloid β antibody or antigen-binding fragment thereof, and is administered in an infusion volume of 20 mL, and with a flow rate of about 2 mL/min. In some embodiments, the first dose is administered on Day 1 and the second dose is administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein:
(a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with a permeation enhancer at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 1000 U/mL. In some embodiments, the first dose and permeation enhancer (at a dose of about 2000 U/mL) are administered on Day 1 and the second dose and permeation enhancer e (at a dose of about 1000 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein:
(a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 1000 U/mL. In some embodiments, the first dose and hyaluronidase (at a dose of about 2000 U/mL) are administered on Day 1 and the second dose and hyaluronidase (at a dose of about 1000 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein:
(a) the first dose comprises about 3400 mg of an anti-amyloid β antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL. In some embodiments, the first dose and recombinant human hyaluronidase (at a dose of about 2000 U/mL) are administered on Day 1 and the second dose and recombinant human hyaluronidase (at a dose of about 1000 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 1700 mg of an anti-amyloid β antibody or antigen-binding fragment thereof, and is administered in an infusion volume of 10 mL, and with a flow rate of about 4 mL/min; and (b) the second dose comprises about 3400 mg of an anti-amyloid β antibody or antigen-binding fragment thereof and is administered in an infusion volume of 40 mL and with a flow rate of about 4 mL/min. In some embodiments, the first dose is administered on Day 1 and the second dose is administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with a permeation enhancer at a dose of about 500 U/mL. In some embodiments, the first dose is administered on Day 1 and the second dose and the permeation enhancer are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with hyaluronidase at a dose of about 500 U/mL. In some embodiments, the first dose is administered on Day 1 and the second dose and the hyaluronidase are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL, and with a flow rate of about 4 mL/min; and (b) the second dose comprises about 6800 mg of an anti-amyloid β antibody or antigen-binding fragment thereof, and is administered at an infusion volume of 40 mL, and with a flow rate of about 4 mL/min; wherein said second dose is co-administered with recombinant human hyaluronidase at a dose of about 500 U/mL. In some embodiments, the first dose is administered on Day 1 and the second dose and the recombinant human hyaluronidase are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with a permeation enhancer at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with a permeation enhancer at a dose of about 500 U/mL. In some embodiments, the first dose and permeation enhancer (at a dose of about 2000 U/mL) are administered on Day 1 and the second dose and permeation enhancer (at a dose of about 500 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with hyaluronidase at a dose of about 500 U/mL. In some embodiments, the first dose and hyaluronidase (at a dose of about 2000 U/mL) are administered on Day 1 and the second dose and hyaluronidase (at a dose of about 500 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a first dose and second dose wherein: (a) the first dose comprises about 3400 mg of an anti-amyloid β antibody or antigen-binding fragment thereof, and is administered in an infusion volume of 20 mL and with a flow rate of about 4 mL/min; wherein said first dose is co-administered with recombinant human hyaluronidase at a dose of about 2000 U/mL; and (b) the second dose comprises about 6800 mg of an anti-amyloid β antibody or antigen-binding fragment thereof, and is administered in an infusion volume of 40 mL, and with a flow rate of about 4 mL/min; wherein said second dose is co-administered with recombinant human hyaluronidase at a dose of about 500 U/mL. In some embodiments, the first dose and recombinant human hyaluronidase (at a dose of about 2000 U/mL) are administered on Day 1 and the second dose and recombinant human hyaluronidase (at a dose of about 500 U/mL) are administered on Day 15.

In some embodiments, the disclosure provides a method of slowing clinical decline in a patient diagnosed with early or mild to moderate Alzheimer's Disease (AD), the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL of a permeation enhancer.

In some embodiments, the disclosure provides a method of slowing clinical decline in a patient diagnosed with early or mild to moderate Alzheimer's Disease (AD), the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL hyaluronidase.

In some embodiments, the disclosure provides a method of slowing clinical decline in a patient diagnosed with early or mild to moderate Alzheimer's Disease (AD), the method comprising subcutaneously administering to the subject a composition comprising about 6800 mg of an anti-amyloid β antibody or antigen-binding fragment thereof, administered in an infusion volume of 40 mL and with a flow rate of about 4 mL/min; and a dose of about 500 U/mL recombinant human hyaluronidase.

In any of the foregoing embodiments, the anti-amyloid β antibody antibody or antigen-binding fragment thereof may be a full-length anti-amyloid β antibody antibody.

In some embodiments, the Alzheimer's disease is autosomal-dominant Alzheimer's disease. In some embodiments, the autosomal-dominant Alzheimer's disease is prodromal, mild, moderate, or mild-to-moderate. In some embodiments, the autosomal-dominant Alzheimer's disease is mild-to-moderate. In some embodiments, the Alzheimer's disease is sporadic AD. In some embodiments, the Alzheimer's disease is early or mild AD.

Any brain targeting antibody or antigen binding fragment thereof may be used in the methods, compositions and uses disclosed herein. In some embodiments, the brain targeting antibody or antigen binding fragment is an anti-amyloid β antibody or antigen binding fragment thereof as disclosed herein. In some embodiments, the anti-amyloid β antibody is crenezumab.

In one aspect, the disclosure provides the use of a composition for the preparation of a medicament for treating Alzheimer's disease in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen binding fragment is an anti-amyloid β antibody or antigen binding fragment thereof as disclosed herein. In some embodiments, the anti-amyloid β antibody is crenezumab.

In one aspect, the disclosure provides the use of a composition for the preparation of a medicament for treating a subject at risk for Alzheimer's disease, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen binding fragment is an anti-amyloid β antibody or antigen binding fragment thereof as disclosed herein. In some embodiments, the anti-amyloid β antibody is crenezumab.

In one aspect, the disclosure provides the use of a composition for the preparation of a medicament for treating cognitive impairment in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen binding fragment is an anti-amyloid β antibody or antigen binding fragment thereof as disclosed herein. In some embodiments, the anti-amyloid β antibody is crenezumab. In some embodiments, the cognitive impairment is mild cognitive impairment (MCI). In some embodiments, the subject is suffering from Alzheimer's Disease.

In one aspect, the disclosure provides the use of a composition for the preparation of a medicament for reducing cognitive impairment in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the cognitive impairment is mild cognitive impairment (MCI). In some embodiments, the brain targeting antibody or antigen binding fragment is an anti-amyloid β antibody or antigen binding fragment thereof as disclosed herein. In some embodiments, the anti-amyloid β antibody is crenezumab. In some embodiments, the cognitive impairment is mild cognitive impairment (MCI). In some embodiments, the subject is suffering from Alzheimer's Disease.

In one aspect, the disclosure provides the use of a composition for the preparation of a medicament for delaying progression of cognitive impairment in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen binding fragment is an anti-amyloid β antibody or antigen binding fragment thereof as disclosed herein. In some embodiments, the anti-amyloid β antibody is crenezumab. In some embodiments, the cognitive impairment is mild cognitive impairment (MCI). In some embodiments, the subject is suffering from Alzheimer's Disease.

In one aspect, the disclosure provides the use of a composition for the preparation of a medicament for treating early or mild to moderate AD without increasing the risk of an adverse event, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen binding fragment is an anti-amyloid β antibody or antigen binding fragment thereof as disclosed herein. In some embodiments, the anti-amyloid β antibody is crenezumab.

In one aspect, the disclosure provides the use of a composition for the preparation of a medicament for delaying progression Alzheimer's Disease (AD) in a patient diagnosed with early or mild to moderate AD, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen binding fragment is an anti-amyloid β antibody or antigen binding fragment thereof as disclosed herein. In some embodiments, the anti-amyloid β antibody is crenezumab.

In one aspect, the disclosure provides the use of a composition for the preparation of a medicament for slowing clinical decline in a patient diagnosed with early or mild to moderate Alzheimer's Disease (AD), wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen binding fragment is an anti-amyloid β antibody or antigen binding fragment thereof as disclosed herein. In some embodiments, the anti-amyloid β antibody is crenezumab.

In some embodiments, the disclosure provides the use of a first dose and a second dose in the manufacture of one or more medicaments for treating Alzheimer's disease in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for treating Alzheimer's disease in a subject wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments for treating Alzheimer's disease in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments for treating Alzheimer's disease in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for treating Alzheimer's disease in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments for treating Alzheimer's disease in a subject wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament for treating Alzheimer's disease in a subject, wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In some embodiments, the disclosure provides the use of a first dose and a second dose in the manufacture of one or more medicaments for treating cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for treating cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments for treating cognitive impairment in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments for treating cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for treating cognitive impairment in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments for treating cognitive impairment in a subject wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament for treating cognitive impairment in a subject, wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In some embodiments, the disclosure provides the use of a first dose and a second dose in the manufacture of one or more medicaments treating a subject at risk for Alzheimer's disease, wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments treating a subject at risk for Alzheimer's disease, wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments treating a subject at risk for Alzheimer's disease, wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments treating a subject at risk for Alzheimer's disease, wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments treating a subject at risk for Alzheimer's disease, wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments treating a subject at risk for Alzheimer's disease, wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament treating a subject at risk for Alzheimer's disease, wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In some embodiments, the disclosure provides the use of a first dose and a second dose in the manufacture of one or more medicaments for reducing cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for reducing cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments for reducing cognitive impairment in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments for reducing cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for reducing cognitive impairment in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments for reducing cognitive impairment in a subject wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament for reducing cognitive impairment in a subject, wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In some embodiments, the disclosure provides the use of a first dose and a second dose in the manufacture of one or more medicaments for delaying progression of cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for delaying progression of cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments for delaying progression of cognitive impairment in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments for delaying progression of cognitive impairment in a subject wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for delaying progression of cognitive impairment in a subject wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments for delaying progression of cognitive impairment in a subject wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament for delaying progression of cognitive impairment in a subject, wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In some embodiments, the disclosure provides the use of a first dose and a second dose in the manufacture of one or more medicaments for delaying progression in a subject diagnosed with early or mild to moderate Alzheimer's Disease (AD) wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for delaying progression in a subject diagnosed with early or mild to moderate Alzheimer's Disease (AD) wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments for delaying progression in a subject diagnosed with early or mild to moderate Alzheimer's Disease (AD) wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments for delaying progression in a subject diagnosed with early or mild to moderate Alzheimer's Disease (AD) wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for delaying progression in a subject diagnosed with early or mild to moderate Alzheimer's Disease (AD) wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments for delaying progression in a subject diagnosed with early or mild to moderate Alzheimer's Disease (AD) wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament for delaying progression in a subject diagnosed with early or mild to moderate Alzheimer's Disease (AD), wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In some embodiments, the disclosure provides the use of a first dose and a second dose in the manufacture of one or more medicaments for treating early or mild to moderate AD without increasing the risk of an adverse event wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for treating early or mild to moderate AD without increasing the risk of an adverse event wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments for treating early or mild to moderate AD without increasing the risk of an adverse event wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments for treating early or mild to moderate AD without increasing the risk of an adverse event wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for treating early or mild to moderate AD without increasing the risk of an adverse event wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments for treating early or mild to moderate AD without increasing the risk of an adverse event wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament for treating early or mild to moderate AD without increasing the risk of an adverse event, wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In some embodiments, the disclosure provides the use of a first dose and a second dose in the manufacture of one or more medicaments for slowing clinical decline in a patient diagnosed with early or mild to moderate Alzheimer's Disease (AD) wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for slowing clinical decline in a patient diagnosed with early or mild to moderate Alzheimer's Disease (AD) wherein:
(a) the first dose comprises about 1700 mg of a brain targeting antibody or antigen-binding fragment thereof, and is suitable for administration subcutaneously in an infusion volume of 10 mL, and with a flow rate of about 2 mL/min; and
(b) the second dose comprises about 3400 mg of a brain targeting antibody or antigen-binding fragment thereof, and is suitable for administration subcutaneously in an infusion volume of 20 mL, and with a flow rate of about 2 mL/min.

In some embodiments, the disclosure provides the use of a first dose and a second dose in the manufacture of one or more medicaments for slowing clinical decline in a patient diagnosed with early or mild to moderate Alzheimer's Disease (AD) wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL.

In some embodiments, the disclosure provides the use of a first dose and a second dose in the manufacture of one or more medicaments for slowing clinical decline in a patient diagnosed with early or mild to moderate Alzheimer's Disease (AD) wherein:
(a) the first dose comprises about 1700 mg of a brain targeting antibody or antigen-binding fragment thereof, and is suitable for administration subcutaneously in an infusion volume of 10 mL, and with a flow rate of about 4 mL/min; and
(b) the second dose comprises about 3400 mg of a brain targeting antibody or antigen-binding fragment thereof and is suitable for administration subcutaneously in an infusion volume of 40 mL and with a flow rate of about 4 mL/min.

In some embodiments, the disclosure provides the use of a first dose and a second dose in the manufacture of one or more medicaments for slowing clinical decline in a patient diagnosed with early or mild to moderate Alzheimer's Disease (AD) wherein:
(a) the first dose comprises about 3400 mg of a brain targeting antibody or antigen-binding fragment thereof, and is suitable for administration subcutaneously in an infusion volume of 20 mL, and with a flow rate of about 4 mL/min; and
(b) the second dose comprises about 6800 mg of a brain targeting antibody or antigen-binding fragment thereof, and is suitable for administration subcutaneously at an infusion volume of 40 mL, and with a flow rate of about 4 mL/min; wherein said second dose is co-administered with a permeation enhancer (e.g., hyaluronidase, including recombinant human hyaluronidase) at a dose of about 500 U/mL.

In some embodiments, the disclosure provides the use of a first dose and second dose in the manufacture of one or more medicaments for slowing clinical decline in a patient diagnosed with early or mild to moderate Alzheimer's Disease (AD) wherein:
(a) the first dose comprises about 3400 mg of a brain targeting antibody or antigen-binding fragment thereof, and is suitable for administration subcutaneously in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration subcutaneously in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL.

In some embodiments, the disclosure provides the use of a composition comprising about 6800 mg of a brain targeting antibody or antigen-binding fragment thereof in the manufacture of a medicament for slowing clinical decline in a patient diagnosed with early or mild to moderate Alzheimer's Disease (AD), wherein the medicament is suitable for administration subcutaneously to the subject in an infusion volume of 40 mL and with a flow rate of about 4 mL/min; wherein the medicament is suitable for co-administering the brain targeting antibody or antigen-binding fragment thereof with a permeation enhancer (e.g., hyaluronidase, including recombinant human hyaluronidase) at a dose of about 500 U/mL.

In any of the foregoing embodiments, the brain targeting antibody or antigen-binding fragment thereof may be a full-length brain targeting antibody. In any of the foregoing embodiments, the brain targeting antibody or antigen-binding fragment thereof may be an anti-amyloid β antibody or antigen binding fragment thereof as disclosed herein. In some embodiments, the anti-amyloid β antibody or antigen-binding fragment thereof is a full-length anti-amyloid β antibody. In some embodiments, the anti-amyloid β antibody is crenezumab. In any of the foregoing embodiments, the brain targeting antibody may be crenezumab.

### Compositions

The present disclosure provides compositions comprising a high volume and a high dose of a brain targeting antibody or antigen-binding fragment thereof that are suitable for subcutaneous administration. Thus, in one aspect, the disclosure provides a composition suitable for administering a high volume and high dose of a brain targeting antibody or antigen binding fragment thereof.

In one aspect, the disclosure provides a composition suitable for administering a high volume and high dose of an anti-amyloid β antibody or antigen binding fragment thereof.

In one aspect, the disclosure provides a composition suitable for administering a high volume and high dose of crenezumab.

In one aspect, the disclosure provides a composition comprising about 400 mg to about 7500 mg of a brain targeting antibody or antigen binding fragment thereof.

In one aspect, the disclosure provides a composition comprising about 600 mg to about 7200 mg of a brain targeting antibody or antigen binding fragment thereof. In some embodiments, the composition comprises about 400 mg of the brain targeting antibody or antigen binding fragment thereof. In some embodiments, the composition comprises about 500 mg of the brain targeting antibody or antigen binding fragment thereof. In some embodiments, the composition comprises about 600 mg of the brain targeting antibody or antigen binding fragment thereof. In some embodiments, the composition comprises about 1200 mg of the brain targeting antibody or antigen binding fragment thereof. In some embodiments, the composition comprises about 1700 mg the brain targeting antibody or antigen binding fragment thereof. In some embodiments, the composition about 1800 mg comprises the brain targeting antibody or antigen binding fragment thereof. In some embodiments, the composition comprises about 2400 mg the brain targeting antibody or antigen-binding fragment thereof. In some embodiments, the composition comprises about 3400 mg the brain targeting antibody or antigen binding fragment thereof. In some embodiments, the composition comprises about 3600 mg the brain targeting antibody or antigen binding fragment thereof. In some embodiments, the composition about 4320 mg comprises the brain targeting antibody or antigen binding fragment thereof. In some embodiments, the composition comprises about 5760 mg the brain targeting antibody or antigen binding fragment thereof. In some embodiments, the composition comprises about 6800 mg the brain targeting antibody or antigen binding fragment thereof. In some embodiments, the composition comprises about 7200 mg the brain targeting antibody or antigen binding fragment thereof. In some embodiments, the composition comprises about 7300 mg of the brain targeting antibody or antigen binding fragment thereof. In some embodiments, the composition comprises about 7400 mg of the brain targeting antibody or antigen binding fragment thereof. In some embodiments, the composition comprises about 7500 mg of the brain targeting antibody or antigen binding fragment thereof.

In some embodiments, the composition further comprises a permeation enhancer. In some embodiments, the permeation enhancer is hyaluronidase (e.g., Amphadase®, Hydase®, Hylenex® and Vitrase®). In some embodiments, the recombinant human hyaluronidase is a human soluble PH20 hyaluronidase glycoprotein, such as rHuPH20.

In some embodiments, the permeation enhancer is about 500 U/mL to about 2000 U/mL. In some embodiments, the concentration of the permeation enhancer is about 500 U/mL. In some embodiments, the concentration of the permeation enhancer is about 1000 U/mL. In some embodiments, the concentration of the permeation enhancer is about 2000 U/mL.

In some embodiments, the permeation enhancer is hyaluronidase. In some embodiments, the hyaluronidase is about 500 U/mL to about 2000 U/mL. In some embodiments, the concentration of the hyaluronidase is about 500 U/mL. In some embodiments, the concentration of the hyaluronidase is about 1000 U/mL. In some embodiments, the concentration of the hyaluronidase is about 2000 U/mL.

In some embodiments, the hyaluronidase is a recombinant human hyaluronidase. In some embodiments, the recombinant human hyaluronidase is about 500 U/mL to about 2000 U/mL. In some embodiments, the concentration of the recombinant human hyaluronidase is about 500 U/mL. In some embodiments, the concentration of the recombinant human hyaluronidase is about 1000 U/mL. In some embodiments, the concentration of the recombinant human hyaluronidase is about 2000 U/mL.

In some embodiments, the brain targeting antibody or antigen-binding fragment thereof and the permeation enhancer are in the same composition. In some embodiments, the brain targeting antibody or antigen-binding fragment thereof and the permeation enhancer are in separate compositions.

In some embodiments, the disclosure provides a composition comprising about 130 mg/mL to about 200 mg/mL of a brain targeting antibody or antigen-binding fragment thereof.

In some embodiments, the concentration of the brain targeting antibody or antigen-binding fragment thereof is about 140 mg/mL to about 190 mg/mL. In some embodiments, the concentration of the brain targeting antibody or antigen-binding fragment thereof is about 150 mg/mL to about 180 mg/mL. In some embodiments, the concentration of the brain targeting antibody or antigen-binding fragment thereof is about 150 mg/mL. In some embodiments, the concentration of the brain targeting antibody or antigen-binding fragment thereof is about 170 mg/mL. In some embodiments, the concentration of the brain targeting antibody or antigen-binding fragment thereof is about 180 mg/mL.

In some embodiments, the composition is suitable for administering the brain targeting antibody or antigen-binding fragment thereof with an infusion volume of about 4 mL to about 60 mL. In some embodiments, the infusion volume is about 10 mL to about 40 mL. In some embodiments, the infusion volume is about 4 mL. In some embodiments, the infusion volume is about 8 mL. In some embodiments, the infusion volume is about 10 mL. In some embodiments, the infusion volume is about 12 mL. In some embodiments, the infusion volume is about 16 mL. In some embodiments, the infusion volume is about 20 mL. In some embodiments, the infusion volume is about 24 mL. In some embodiments, the infusion volume is about 32 mL. In some embodiments, the infusion volume is about 40 mL.

In some embodiments, the composition is suitable for administering the brain targeting antibody or antigen-binding fragment thereof with a flow rate of about 1 mL/min to about 5 mL/min. In some embodiments, the flow rate is about 2 mL/min to about 4 mL/min. In some embodiments, the flow rate is about 2 mL/min. In some embodiments, the flow rate is about 4 mL/min.

In one aspect, the disclosure provides a composition comprising about 400 mg to about 7500 mg of an anti-amyloid β antibody or antigen binding fragment thereof.

In one aspect, the disclosure provides a composition comprising about 600 mg to about 7200 mg of an anti-amyloid β antibody or antigen binding fragment thereof. In some embodiments, the composition comprises about 400 mg of the anti-amyloid β antibody or antigen binding fragment thereof. In some embodiments, the composition comprises about 500 mg of the anti-amyloid β antibody or antigen binding fragment thereof. In some embodiments, the composition comprises about 600 mg of the anti-amyloid β antibody or antigen binding fragment thereof. In some embodiments, the composition comprises about 1200 mg of the anti-amyloid β antibody or antigen binding fragment thereof. In some embodiments, the composition comprises about 1700 mg the anti-amyloid β antibody or antigen binding fragment thereof. In some embodiments, the composition about 1800 mg comprises the anti-amyloid β antibody or antigen binding fragment thereof. In some embodiments, the composition comprises about 2400 mg the anti-amyloid β antibody or antigen-binding fragment thereof. In some embodiments, the composition comprises about 3400 mg the anti-amyloid β antibody or antigen binding fragment thereof. In some embodiments, the composition comprises about 3600 mg the anti-amyloid β antibody or antigen binding fragment thereof. In some embodiments, the composition about 4320 mg comprises the anti-amyloid β antibody or antigen binding fragment thereof. In some embodiments, the composition comprises about 5760 mg the anti-amyloid β antibody or antigen binding fragment thereof. In some embodiments, the composition comprises about 6800 mg the anti-amyloid β antibody or antigen binding fragment thereof. In some embodiments, the composition comprises about 7200 mg the anti-amyloid β antibody or antigen binding fragment thereof. In some embodiments, the composition comprises about 7300 mg of the anti-amyloid β antibody or antigen binding fragment thereof. In some embodiments, the composition comprises about 7400 mg of the anti-amyloid β antibody or antigen binding fragment thereof. In some embodiments, the composition comprises about 7500 mg of the anti-amyloid β antibody or antigen binding fragment thereof.

In some embodiments, the composition further comprises a permeation enhancer. In some embodiments, the permeation enhancer is hyaluronidase (e.g., Amphadase®, Hydase®, Hylenex® and Vitrase®). In some embodiments, the permeation enhancer is recombinant human hyaluronidase. In some embodiments, the recombinant human hyaluronidase is a human soluble PH20 hyaluronidase glycoprotein, such as rHuPH20.

In some embodiments, the permeation enhancer is about 500 U/mL to about 2000 U/mL. In some embodiments, the concentration of the permeation enhancer is about 500 U/mL. In some embodiments, the concentration of permeation enhancer is about 1000 U/mL. In some embodiments, the concentration of permeation enhancer is about 2000 U/mL.

In some embodiments, the composition further comprises hyaluronidase. In some embodiments, the hyaluronidase is about 500 U/mL to about 2000 U/mL. In some embodiments, the concentration of the hyaluronidase is about 500 U/mL. In some embodiments, the concentration of the hyaluronidase is about 1000 U/mL. In some embodiments, the concentration of the hyaluronidase is about 2000 U/mL.

In some embodiments, the composition further comprises recombinant human hyaluronidase. In some embodiments, the recombinant human hyaluronidase is about 500 U/mL to about 2000 U/mL. In some embodiments, the concentration of the recombinant human hyaluronidase is about 500 U/mL. In some embodiments, the concentration of the recombinant human hyaluronidase is about 1000 U/mL. In some embodiments, the concentration of the recombinant human hyaluronidase is about 2000 U/mL.

In some embodiments, the anti-amyloid β antibody or antigen-binding fragment thereof and the permeation enhancer are in the same composition. In some embodiments, the anti-amyloid β antibody or antigen-binding fragment thereof and the permeation enhancer are in separate compositions.

In some embodiments, the disclosure provides a composition comprising about 130 mg/mL to about 200 mg/mL of an anti-amyloid β antibody or antigen-binding fragment thereof.

In some embodiments, the concentration of the anti-amyloid β antibody or antigen-binding fragment thereof is about 140 mg/mL to about 190 mg/mL. In some embodiments, the concentration of the anti-amyloid β antibody or antigen-binding fragment thereof is about 150 mg/mL to about 180 mg/mL. In some embodiments, the concentration of the anti-amyloid β antibody or antigen-binding fragment thereof is about 150 mg/mL. In some embodiments, the concentration of the anti-amyloid β antibody or antigen-binding fragment thereof is about 170 mg/mL. In some embodiments, the concentration of the anti-amyloid β antibody or antigen-binding fragment thereof is about 180 mg/mL.

In some embodiments, the composition is suitable for administering the anti-amyloid β antibody or antigen-binding fragment thereof with an infusion volume of about 4 mL to about 60 mL. In some embodiments, the infusion volume is about 10 mL to about 40 mL. In some embodiments, the infusion volume is about 4 mL. In some embodiments, the infusion volume is about 8 mL. In some embodiments, the infusion volume is about 10 mL. In some embodiments, the infusion volume is about 12 mL. In some embodiments, the infusion volume is about 16 mL. In some embodiments, the infusion volume is about 20 mL. In some embodiments, the infusion volume is about 24 mL. In some embodiments, the infusion volume is about 32 mL. In some embodiments, the infusion volume is about 40 mL.

In some embodiments, the composition is suitable for administering the anti-amyloid β antibody or antigen-binding fragment thereof with a flow rate of about 1 mL/min to about 5 mL/min. In some embodiments, the flow rate is about 2 mL/min to about 4 mL/min. In some embodiments, the flow rate is about 2 mL/min. In some embodiments, the flow rate is about 4 mL/min.

In some embodiments, the anti-amyloid β antibody or antigen-binding fragment thereof comprises: (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6.

In some embodiments, the anti-amyloid β antibody or antigen-binding fragment thereof comprises a VH domain comprising the amino acid sequence of SEQ ID NO: 7.

In some embodiments, the anti-amyloid β antibody or antigen-binding fragment thereof comprises a VL domain comprising the amino acid sequence of SEQ ID NO: 8. In some embodiments, the anti-amyloid β antibody comprises a heavy chain comprising the amino acid sequence of SEQ ID NO: 9. In some embodiments, the anti-amyloid β antibody comprises a light chain comprising the amino acid sequence of SEQ ID NO: 10. In some embodiments, the anti-amyloid β antibody is crenezumab.

In one aspect, the disclosure provides a composition for use in treating Alzheimer's disease in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen-binding fragment thereof is an anti-amyloid β antibody or antigen binding fragment thereof. In some embodiments, the anti-amyloid β antibody is crenezumab.

In one aspect, the disclosure provides a composition for use in treating a subject at risk for Alzheimer's disease, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen-binding fragment thereof is an anti-amyloid β antibody or antigen binding fragment thereof. In some embodiments, the anti-amyloid β antibody is crenezumab.

In one aspect, the disclosure provides a composition for use in treating cognitive impairment in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen-binding fragment thereof is an anti-amyloid β antibody or antigen binding fragment thereof. In some embodiments, the anti-amyloid β antibody is crenezumab. In some embodiments, the cognitive impairment is mild cognitive impairment (MCI). In some embodiments, the subject is suffering from Alzheimer's Disease.

In one aspect, the disclosure provides a composition for use in reducing cognitive impairment in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/ml to about 200 mg/mL. In some embodiments, the cognitive impairment is mild cognitive impairment (MCI). In some embodiments, the brain targeting antibody or antigen-binding fragment thereof is an anti-amyloid β antibody or antigen binding fragment thereof. In some embodiments, the anti-amyloid β antibody is crenezumab. In some embodiments, the cognitive impairment is mild cognitive impairment (MCI). In some embodiments, the subject is suffering from Alzheimer's Disease.

In one aspect, the disclosure provides a composition for use in delaying progression of cognitive impairment in a subject, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen-binding fragment thereof is an anti-amyloid β antibody or antigen binding fragment thereof. In some embodiments, the anti-amyloid β antibody is crenezumab. In some embodiments, the cognitive impairment is mild cognitive impairment (MCI). In some embodiments, the subject is suffering from Alzheimer's Disease.

In one aspect, the disclosure provides a composition for use in treating early or mild to moderate AD without increasing the risk of an adverse event, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen-binding fragment thereof is an anti-amyloid β antibody or antigen binding fragment thereof. In some embodiments, the anti-amyloid β antibody is crenezumab.

In one aspect, the disclosure provides a composition for use in delaying progression of Alzheimer's Disease (AD) in a patient diagnosed with early or mild to moderate AD, wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen-binding fragment thereof is an anti-amyloid β antibody or antigen binding fragment thereof. In some embodiments, the anti-amyloid β antibody is crenezumab.

In one aspect, the disclosure provides a composition for use in slowing clinical decline in a patient diagnosed with early or mild to moderate Alzheimer's Disease (AD), wherein the composition is suitable for subcutaneous administration and comprises a brain targeting antibody or antigen-binding fragment thereof, wherein the brain targeting antibody or antigen-binding fragment thereof is at a concentration of about 130 mg/mL to about 200 mg/mL. In some embodiments, the brain targeting antibody or antigen-binding fragment thereof is an anti-amyloid β antibody or antigen binding fragment thereof. In some embodiments, the anti-amyloid β antibody is crenezumab.

In some embodiments, the disclosure provides a composition comprising a first dose and a second dose for use in treating Alzheimer's disease in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in treating Alzheimer's disease in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with recombinant human hyaluronidase at a dose of about 1000 U/mL. In some embodiments, the first dose and a permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in treating Alzheimer's disease in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in treating Alzheimer's disease in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in treating Alzheimer's disease in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and (b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in treating Alzheimer's disease in a subject, wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in treating cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in treating cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and (b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in treating cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in treating cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in treating cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and (b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in treating cognitive impairment in a subject, wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in treating a subject at risk for Alzheimer's disease, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in treating a subject at risk for Alzheimer's disease, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and a permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in treating a subject at risk for Alzheimer's disease, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in treating a subject at risk for Alzheimer's disease, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in treating a subject at risk for Alzheimer's disease, wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in treating a subject at risk for Alzheimer's disease, wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in reducing cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in reducing cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in reducing cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in reducing cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in reducing cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in reducing cognitive impairment in a subject, wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in delaying progression of cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in delaying progression of cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in delaying progression of cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in delaying progression of cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in delaying progression of cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in delaying progression of cognitive impairment in a subject, wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in delaying progression of cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in delaying progression of cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in delaying progression of cognitive impairment in a subject, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in delaying progression of cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in delaying progression of cognitive impairment in a subject, wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in delaying progression of cognitive impairment in a subject, wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in delaying progression in a subject diagnosed with early or mild to moderate Alzheimer's Disease (AD), wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in delaying progression in a subject diagnosed with early or mild to moderate Alzheimer's Disease (AD), wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in delaying progression in a subject diagnosed with early or mild to moderate Alzheimer's Disease (AD), wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in delaying progression in a subject diagnosed with early or mild to moderate Alzheimer's Disease (AD), wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in delaying progression in a subject diagnosed with early or mild to moderate Alzheimer's Disease (AD), wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and (b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and a permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in delaying progression in a subject diagnosed with early or mild to moderate Alzheimer's Disease (AD), wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in treating early or mild to moderate AD without increasing the risk of an adverse event, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in treating early or mild to moderate AD without increasing the risk of an adverse event, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and (b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and permeation enhancer (e.g., hyaluronidase, including recombinant human hyaluronidase) (at a dose of about 2000 U/mL) are administered on Day 1 and the second dose permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in treating early or mild to moderate AD without increasing the risk of an adverse event, wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in treating early or mild to moderate AD without increasing the risk of an adverse event, wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in treating early or mild to moderate AD without increasing the risk of an adverse event, wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and (b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in treating early or mild to moderate AD without increasing the risk of an adverse event, wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In some embodiments, the disclosure provides a composition comprising a first dose and a second dose for use in slowing clinical decline in a patient diagnosed with early or mild to moderate Alzheimer's Disease (AD), wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 2 mL/min; and (b) the second dose comprises about 3400 mg of a brain targeting antibody or antigen-binding fragment thereof, and is suitable for administration in an infusion volume of 20 mL, and with a flow rate of about 2 mL/min.

In some embodiments, the disclosure provides a composition comprising a first dose and a second dose for use in slowing clinical decline in a patient diagnosed with early or mild to moderate Alzheimer's Disease (AD), wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; wherein said first dose is for co-administration with a permeation enhancer (e.g., hyaluronidase, including recombinant human hyaluronidase) at a dose of about 2000 U/mL; and
(b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 1000 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 1000 U/mL) are administered on Day 15.

In some embodiments, the disclosure provides a composition comprising a first dose and a second dose for use in slowing clinical decline in a patient diagnosed with early or mild to moderate Alzheimer's Disease (AD), wherein the composition is suitable for subcutaneous administration, and wherein: (a) the first dose comprises about 1700 mg of a brain targeting antibody or antigen-binding fragment thereof, and is suitable for administration in an infusion volume of 10 mL, and with a flow rate of about 4 mL/min; and (b) the second dose comprises about 3400 mg of a brain targeting antibody or antigen-binding fragment thereof and is suitable for administration in an infusion volume of 40 mL and with a flow rate of about 4 mL/min. In some embodiments, the first dose is administered on Day 1 and the second dose is administered on Day 15.

In some embodiments, the disclosure provides a composition comprising a first dose and a second dose for use in slowing clinical decline in a patient diagnosed with early or mild to moderate Alzheimer's Disease (AD), wherein the composition is suitable for subcutaneous administration, and wherein: (a) the first dose comprises about 3400 mg of a brain targeting antibody or antigen-binding fragment thereof, and is suitable for administration in an infusion volume of 20 mL, and with a flow rate of about 4 mL/min; and (b) the second dose comprises about 6800 mg of a brain targeting antibody or antigen-binding fragment thereof, and is suitable for administration at an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose is administered on Day 1 and the second dose and the permeation enhancer (e.g., hyaluronidase, including recombinant human hyaluronidase) are administered on Day 15.

In some embodiments, the disclosure provides a composition comprising a first dose and a second dose for use in slowing clinical decline in a patient diagnosed with early or mild to moderate Alzheimer's Disease (AD), wherein the composition is suitable for subcutaneous administration, and wherein: (a) the first dose comprises about 3400 mg of a brain targeting antibody or antigen-binding fragment thereof, and is suitable for administration in an infusion volume of 20 mL and with a flow rate of about 4 mL/min; wherein said first dose is co-administered with a permeation enhancer (e.g., hyaluronidase, including recombinant human hyaluronidase) at a dose of about 2000 U/mL; and (b) the second dose comprises about 6800 mg of a brain targeting antibody or antigen-binding fragment thereof, and is suitable for administration in an infusion volume of 40 mL, and with a flow rate of about 4 mL/min; wherein said second dose is for co-administration with a permeation enhancer (e.g., hyaluronidase, including recombinant human hyaluronidase) at a dose of about 500 U/mL. In some embodiments, the first dose and permeation enhancer (e.g., hyaluronidase, including recombinant human hyaluronidase) (at a dose of about 2000 U/mL) are administered on Day 1 and the second dose and permeation enhancer (e.g., hyaluronidase, including recombinant human hyaluronidase) (at a dose of about 500 U/mL) are administered on Day 15.

In some embodiments, the disclosure provides a composition for use in slowing clinical decline in a patient diagnosed with early or mild to moderate Alzheimer's Disease (AD), wherein the composition is suitable for subcutaneous administration, and wherein the composition comprises about 6800 mg of a brain targeting antibody or antigen-binding fragment thereof, is suitable for administration in an infusion volume of 40 mL and with a flow rate of about 4 mL/min; and a dose of about 500 U/mL of a permeation enhancer (e.g., hyaluronidase, including recombinant human hyaluronidase).

In any of the foregoing embodiments, the brain targeting antibody or antigen-binding fragment thereof may be a full-length brain targeting antibody. In any of the foregoing embodiments, the brain targeting antibody or antigen-binding fragment thereof may be an anti-amyloid β antibody or antigen binding fragment thereof. In some embodiments, the anti-amyloid β antibody or antigen binding fragment thereof is a full-length anti-amyloid β antibody. In some embodiments, the anti-amyloid β antibody is crenezumab. In any of the foregoing embodiments, the brain targeting antibody may be crenezumab.

### Exemplary Antibodies

In some embodiments, the methods and compositions comprise a brain-targeting antibody or an antigen-binding fragment thereof. In some embodiments, the brain-targeting antibody or antigen-binding fragment thereof is an anti-amyloid β antibody or antigen-binding fragment thereof. The anti-amyloid β antibodies used in the compositions and methods disclosed herein include the anti-amyloid β antibodies disclosed, e.g., in U.S. Pat. No. 7,892,544 and WO2015/120233, both of which are incorporated by reference herein in their entirety. In some embodiments, the anti-amyloid β antibody is a humanized antibody. In some embodiments, the anti-amyloid β antibody comprises HVRs as in any one of the embodiments of this disclosure, and further comprises an acceptor human framework, e.g., a human immunoglobulin framework or a human consensus framework.

In some embodiments, a humanized antibody or a fragment thereof according to this disclosure, is provided comprising at least one light chain or a fragment thereof or at least one heavy chain or a fragment thereof incorporating at least one, particularly two and more particularly three CDR regions obtained from a mouse donor antibody, particularly from mouse antibody ACI-01-Ab7C2 (named “mC2” and hC2 for the humanized C2 antibody, throughout this disclosure) deposited 1 Dec. 2005 with the “Deutsche Sammlung von Mikroorganismen und Zellkulturen GmbH (DSMZ) in Braunschweig, Mascheroder Weg 1 B, 38124 Braunschweig, under Accession No. DSM ACC2750, wherein said antibody or fragment thereof has an affinity to the Aβ antigen which is at least 5 times, at least 8 times, at least 10 times, or at least 15 times higher than that of the mouse donor antibody.

The antibody of this disclosure can be, in one embodiment, a whole antibody (e.g., with two full length light chains and two full length heavy chains) of any isotype and subtype (e.g., IgM, IgD, IgG1, IgG2, IgG3, IgG4, IgE, IgA1 and IgA2); but especially an antibody of the IgG4 isotype; alternatively, in another embodiment, it can be an antigen-binding fragment (e.g., Fab, F(ab′)2, and Fv) of a whole antibody. In some embodiments, the fragment is selected from the group consisting of a Fab fragment, a Fab′ fragment, a F(ab)2fragment, and a Fv fragment, including the products of an Fab immunoglobulin expression library and epitope-binding fragments of any of the antibodies and fragments mentioned above.

### 1. Antibody Affinity

In some embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M).

In some embodiments, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (I)-labeled antigen in the presence of a titration series of unlabeled antigen, then capturing bound antigen with an anti-Fab antibody-coated plate (see, e.g., Chen et al. et al, J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed, and the plate washed eight times with 0.1% polysorbate 20 (TWEEN-20®) in PBS. When the plates have dried, 150 μL/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, NJ) at 25° C. with immobilized antigen CM5 chips at −10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 L/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL/min. Association rates (kon) and dissociation rates (koff) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al, J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° ° C. of a 20 nM antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al Nat. Med. 9: 129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al, Nat. Med. 9: 129-134 (2003); and Hollinger et al, Proc. Natl. Acad. Sci. USA 90: 6444-6448 (1993). Triabodies and tetrabodies are also described in Hudson et al, Nat. Med. 9: 129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (Domantis, Inc., Waltham, MA; see, e.g., U.S. Pat. No. 6,248,516 B1). In some embodiments, two or more single-domain antibodies may be joined together to form an immunoglobulin construct with multivalent affinity (i.e., the N- or C-terminus of a first single-domain antibody may be fused or otherwise joined to the N- or C-terminus of a second single-domain antibody).

Antibody fragments can be made by various techniques, including but not limited to proteolytic digestion of an intact antibody, as well as, production by recombinant host cells (e.g. E. coli or phage), as described herein.

### 3. Chimeric and Humanized Antibodies

In some embodiments, an antibody provided herein is a chimeric antibody. Certain chimeric antibodies are described, e.g., in U.S. Pat. No. 4,816,567; and Morrison et al, Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13: 1619-1633 (2008), and are further described, e.g., in Riechmann et al, Nature 332:323-329 (1988); Queen et al, Proc. Nat'l Acad. Sci. USA 86: 10029-10033 (1989); U.S. Pat. Nos. 5,821,337, 7,527,791, 6,982,321, and 7,087,409; Kashmiri et al, Methods 36:25-34 (2005) (describing SDR (a-CDR) grafting); Padlan, Mol. Immunol. 28:489-498 (1991) (describing “resurfacing”); Dall'Acqua et al, Methods 36:43-60 (2005) (describing “FR shuffling”); and Osbourn et al, Methods 36:61-68 (2005) and Klimka et al, Br. J. Cancer, 83:252-260 (2000) (describing the “guided selection” approach to FR shuffling).

Human framework regions that may be used for humanization include but are not limited to: framework regions selected using the “best-fit” method (see, e.g., Sims. et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol, 151:2623 (1993)); human mature (somatically mutated) framework regions or human germline framework regions (see, e.g., Almagro and Fransson, Front. Biosci. 13: 1619-1633 (2008)); and framework regions derived from screening FR libraries (see, e.g., Baca et al, J. Biol. Chem. 272: 10678-10684 (1997) and Rosok et al, J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23: 1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol, 133: 3001 (1984); Brodeur et al, Monoclonal Antibody Production Techniques and Applications, pp. 51-63 (Marcel Dekker, Inc., New York, 1987); and Boerner et al, J. Immunol, 147: 86 (1991).) Human antibodies generated via human B-cell hybridoma technology are also described in Li et al, Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of this disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178: 1-37 (O'Brien et al., ed., Human Press, Totowa, N J, 2001) and further described, e.g., in the McCafferty et al., Nature 348:552-554; Clackson et al, Nature 352: 624-628 (1991); Marks et al, J. Mol. Biol. 222: 581-597 (1992); Marks and Bradbury, in Methods in Molecular Biology 248: 161-175 (Lo, ed., Human Press, Totowa, N J, 2003); Sidhu et al, J. Mol. Biol. 338(2): 299-310 (2004); Lee et al, J. Mol. Biol. 340(5): 1073-1093 (2004); Fellouse, Proc. Natl. Acad. Sci. USA 101(34): 12467-12472 (2004); and Lee et al, J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al, EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In some embodiments, an antibody provided herein is a multispecific antibody, e.g., a bispecific antibody. Multispecific antibodies are monoclonal antibodies that have binding specificities for at least two different sites. In some embodiments, one of the binding specificities is for Aβ and the other is for any other antigen. In some embodiments, bispecific antibodies may bind to two different epitopes of Aβ. Bispecific antibodies may also be used to localize cytotoxic agents to cells. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al, EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al, Science, 229: 81 (1985)); using leucine zippers to produce bispecific antibodies (see, e.g., Kostelny et al, J. Immunol, 148(5): 1547-1553 (1992)); using “diabody” technology for making bispecific antibody fragments (see, e.g., Hollinger et al, Proc. Natl. Acad. Sci. USA, 90:6444-6448 (1993)); and using single-chain Fv (sFv) dimers (see, e.g. Gruber et al, J. Immunol, 152:5368 (1994)); and preparing trispecific antibodies as described, e.g., in Tutt et al J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to Aβ as well as another, different antigen (see, US 2008/0069820, for example).

### 7. Antibody Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In some embodiments, antibody variants having one or more amino acid substitutions are provided. Sites of interest for substitutional mutagenesis include the HVRs and FRs. Conservative substitutions are shown in Table 2 under the heading of “conservative substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

TABLE 2
Amino acid substitutions

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Glu | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody. In certain embodiments, affinity matured antibodies will have nanomolar or even picomolar affinities for the target antigen. Affinity matured antibodies are produced by procedures known in the art, including, e.g., using phage display-based affinity maturation techniques such as those described herein. Briefly, one or more HV residues are mutated and the variant antibodies displayed on phage and screened for a particular biological activity (e.g., binding affinity). Other procedures are also known. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci, USA 91:3809-3813 (1994); Schier et al. Gene 169: 147-155 (1996); Yelton et al. J. Immunol. 155: 1994-2004 (1995); Jackson et al, J. Immunol. 154(7):3310-9 (1995); and Hawkins et al. J. Mol. Biol. 226:889-896 (1992).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178: 1-37 (O'Brien et al, ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant VH and VL sequences as provided in this disclosure, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of this disclosure may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65%> or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al, especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al, Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al); U.S. Pat. No. 6,602,684 (Umana et al); and US 2005/0123546 (Umana et al). Antibody variants with at least one galactose residue in the oligosaccharide attached to the Fc region are also provided. Such antibody variants may have improved CDC function. Such antibody variants are described, e.g., in WO 1997/30087 (Patel et al); WO 1998/58964 (Raju, S.); and WO 1999/22764 (Raju, S.).

### Fc Region Variants

In some embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, this disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc RIII only, whereas monocytes express FcλRI, FcλRII and Fc RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al, Proc. Nat'l Acad. Sci. USA 82:1499-1502 (1985); 5,821,337 (see Bruggemann, M. et all, J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al, J. Immunol. Methods 202: 163 (1996); Cragg, M. S. et al, Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al, Int'l. Immunol. 18(12): 1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

### Cysteine Engineered Antibody Variants

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: V205 (Kabat numbering) of the light chain; Al 18 (EU numbering) of the heavy chain; and S400 (EU numbering) of the heavy chain Fc region. Cysteine engineered antibodies may be generated as described, e.g., in U.S. Pat. No. 7,521,541.

### Antibody Derivatives

In some embodiments, an antibody provided herein may be further modified to contain additional non-proteinaceous moieties that are known in the art and readily available. The moieties suitable for derivatization of the antibody include but are not limited to water soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and non-proteinaceous moiety that may be selectively heated by exposure to radiation are provided. In one embodiment, the non-proteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibodies may be produced using recombinant methods and compositions, e.g., as described in U.S. Pat. No. 4,816,567. In some embodiments, isolated nucleic acid encoding an anti-Aβ antibody described herein is provided. Such nucleic acid may encode an amino acid sequence comprising the VL and/or an amino acid sequence comprising the VH of the antibody (e.g., the light and/or heavy chains of the antibody). In some embodiments, one or more vectors (e.g., expression vectors) comprising such nucleic acid are provided. In some embodiments, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NSO, Sp20 cell). In some embodiments, a method of making an anti-Aβ antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-Aβ antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See, e.g., Gerngross, Nat. Biotech. 22: 1409-1414 (2004), and Li et al, Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLA TIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al, J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VEPvO-76); human cervical carcinoma cells (HELA); canine kidney cells (MDCK; buffalo rat liver cells (BRL 3 A); human lung cells (W138); human liver cells (Hep G2); mouse mammary tumor (MMT 060562); TRI cells, as described, e.g., in Mather et al, Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al, Proc. Natl. Acad. Sci. USA 77:4216 (1980)); and myeloma cell lines such as Y0, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Assays

Anti-Aβ antibodies provided herein may be identified, screened for, or characterized for their physical/chemical properties and/or biological activities by various assays known in the art.

### Binding Assays and Other Assays

In one aspect, an antibody of this disclosure is tested for its antigen binding activity, e.g., by known methods such as ELISA, Western blot, etc.

In another aspect, competition assays may be used to identify an antibody that competes with an anti-Aβ antibody of this disclosure for binding to Aβ. In certain embodiments, such a competing antibody binds to the same epitope (e.g., a linear or a conformational epitope) that is bound by crenezumab or another anti-Aβ antibody specified herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized Aβ in the desired form (e.g., monomeric, oligomeric, or fibril) is incubated in a solution comprising a first labeled antibody that binds to Aβ (e.g., crenezumab) and a second unlabeled antibody that is being tested for its ability to compete with the first antibody for binding to Aβ. The second antibody may be present in a hybridoma supernatant. As a control, immobilized Aβ is incubated in a solution comprising the first labeled antibody but not the second unlabeled antibody. After incubation under conditions permissive for binding of the first antibody to Aβ, excess unbound antibody is removed, and the amount of label associated with immobilized Aβ is measured. If the amount of label associated with immobilized Aβ is substantially reduced in the test sample relative to the control sample, then that indicates that the second antibody is competing with the first antibody for binding to Aβ. See Harlow and Lane (1988) Antibodies: A Laboratory Manual ch. 14 (Cold Spring Harbor Laboratory, Cold Spring Harbor, NY).

### Activity Assays

In one aspect, assays are provided for identifying anti-Aβ antibodies thereof having biological activity, for example the biological activity of crenezumab. Biological activity may include, but is not limited to, e.g., prevention of aggregation of monomeric Aβ into oligomeric Aβ, or disaggregation of oligomeric Aβ into monomeric Aβ. Antibodies having such biological activity in vivo and/or in vitro are also provided. In some embodiments, an antibody of this disclosure is tested for such biological activity.

Specific anti-amyloid β antibody sequences are provided in Table 3.

TABLE 3
Amino acid sequences

| SEQ | | |
|---|---|---|
| ID NO. | Name | Amino acid Sequence |
| 1 | HVR-H1 | GFTFSSYGMS |
| 2 | HVR-H2 | SINSNGGSTYYPDSVK |
| 3 | HVR-H3 | GDY |
| 4 | HVR-L1 | RSSQSLVYSNGDTYLH |
| 5 | HVR-L2 | KVSNRFS |
| 6 | HVR-L3 | SQSTHVPWT |
| 7 | VH domain | EVQLVESGGGLVQPGGSLRLSCAASGFTFSSYGMSWVRQAPG |
| | | KGLELVASINSNGGSTYYPDSVKGRFTISRDNAKNSLYLQMNS |
| | | LRAEDTAVYYCASGDYWGQGTTVTVSS |
| 8 | VL domain | DIVMTQSPLSLPVTPGEPASISCRSSQSLVYSNGDTYLHWYLQK |
| | | PGQSPQLLIYKVSNRFSGVPDRFSGSGSGTDFTLKISRVEAEDV |
| | | GVYYCSQSTHVPWTFGQGTKVEIK |
| 9 | Heavy chain | EVQLVESGGGLVQPGGSLRLSCAASGFTFSSYGMSWVRQAPG |
| | | KGLELVASINSNGGSTYYPDSVKGRFTISRDNAKNSLYLQMNS |
| | | LRAEDTAVYYCASGDYWGQGTTVTVSSASTKGPSVFPLAPCS |
| | | RSTSESTAALGCLVKDYFPEPVTVSWNSGALTSGVHTFPAVLQ |
| | | SSGLYSLSSVVTVPSSSLGTKTYTCNVDHKPSNTKVDKRVESK |
| | | YGPPCPPCPAPEFLGGPSVFLFPPKPKDTLMISRTPEVTCVVVD |
| | | VSQEDPEVQFNWYVDGVEVHNAKTKPREEQFNSTYRVVSVLT |
| | | VLHQDWLNGKEYKCKVSNKGLPSSIEKTISKAKGQPREPQVYT |
| | | LPPSQEEMTKNQVSLTCLVKGFYPSDIAVEWESNGQPENNYKT |
| | | TPPVLDSDGSFFLYSRLTVDKSRWQEGNVFSCSVMHEALHNH |
| | | YTQKSLSLSLGK |
| 10 | Light chain | DIVMTQSPLSLPVTPGEPASISCRSSQSLVYSNGDTYLHWYLQK |
| | | PGQSPQLLIYKVSNRFSGVPDRFSGSGSGTDFTLKISRVEAEDV |
| | | GVYYCSQSTHVPWTFGQGTKVEIKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFNR |
| | | GEC |

### EXAMPLES

### Example 1: Study of Safety and Tolerability of Single Ascending Volumes

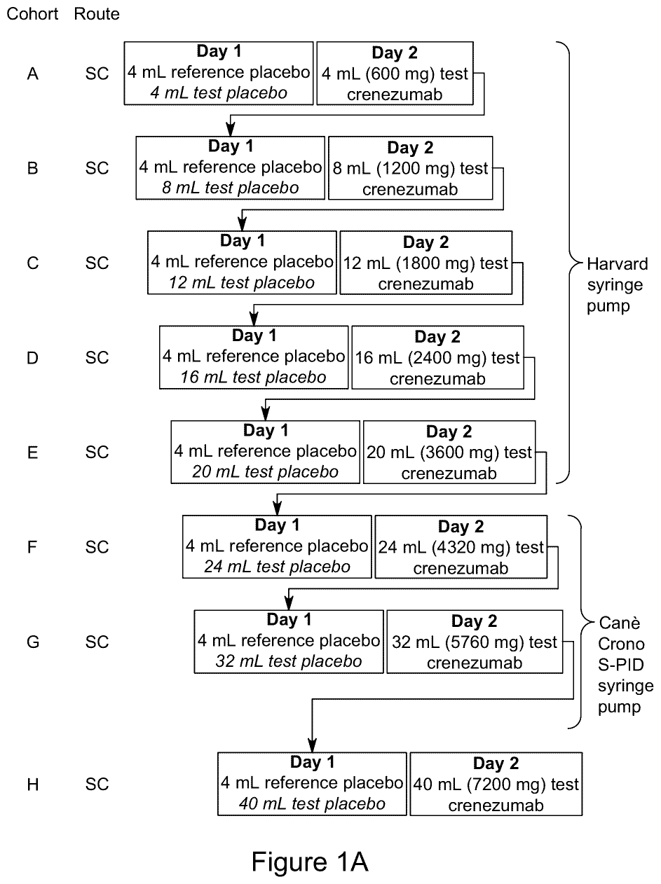

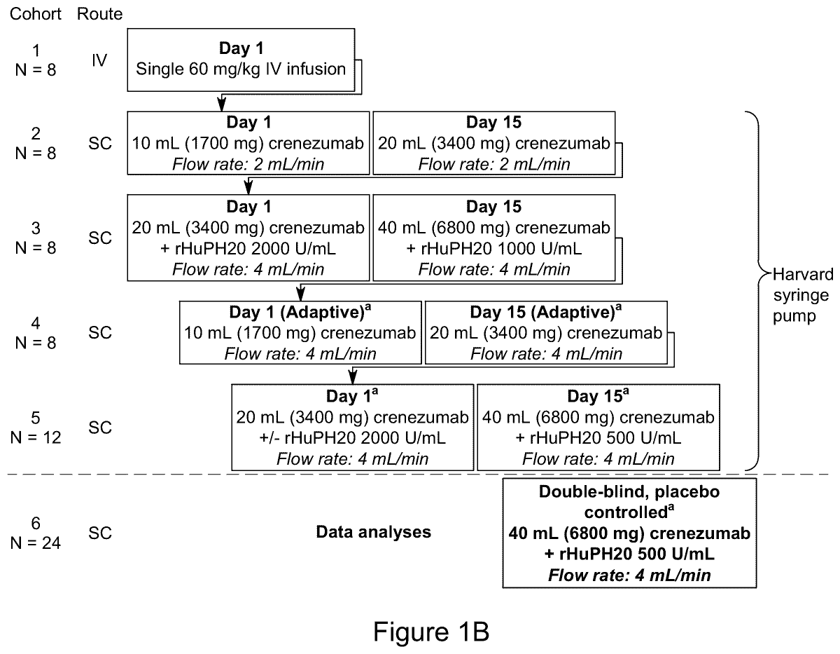

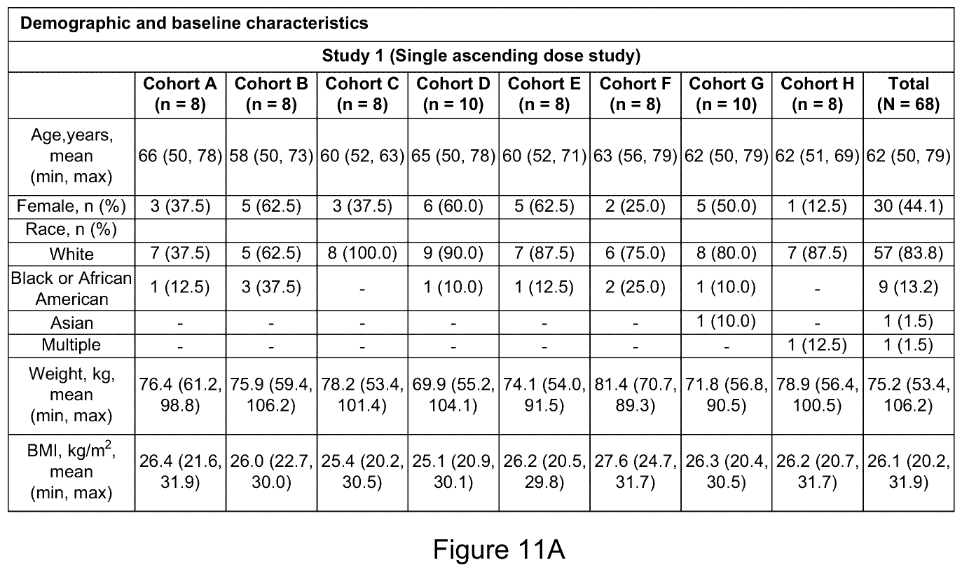

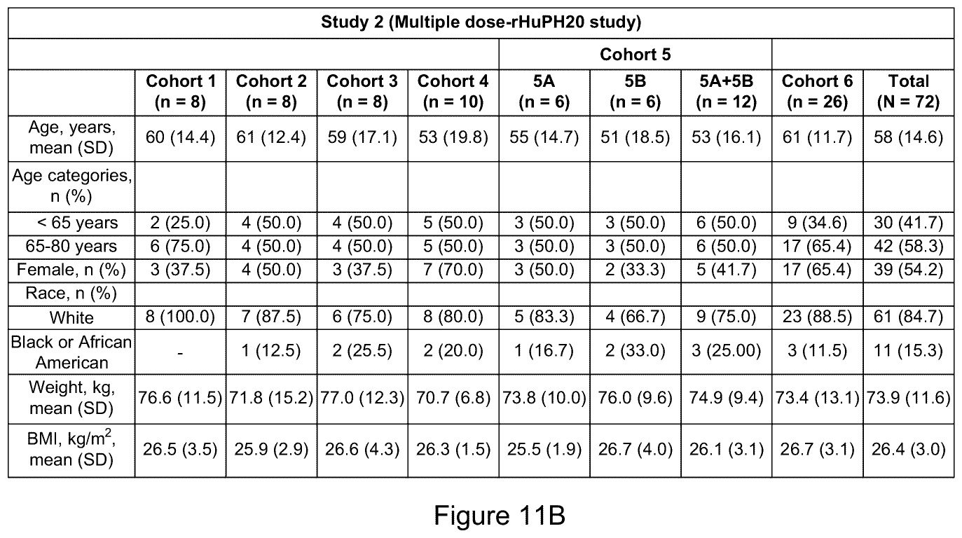

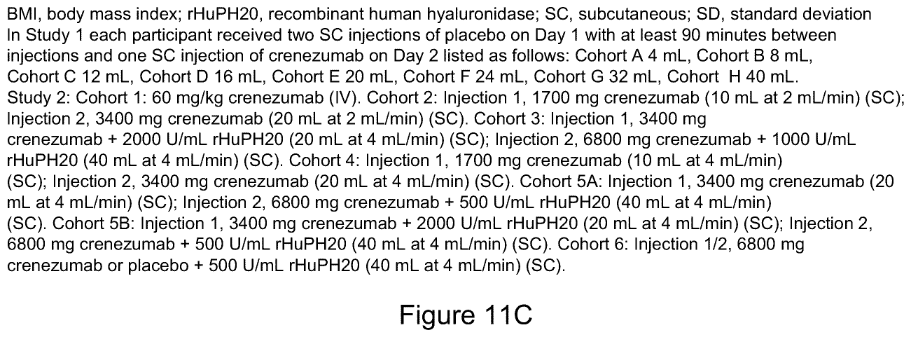

A Phase I study (Study 1) was performed and recruited 68 healthy subjects (64 subjects completed the study) to assess the safety and tolerability of single ascending volumes of subcutaneously administered placebo, and of single ascending doses (e.g., 600-7200 mg) of subcutaneously administered crenezumab. The pharmacokinetics of crenezumab after subcutaneous (SC) infusion and infusion site leakage were also assessed. The subjects were separated into 8 cohorts (e.g., A-H) (FIG. 1A). Subjects were generally well-matched between cohorts in terms of age, weight, body mass index (BMI) and race; gender distribution varied 10 (FIGS. 11A-11C). 67 subjects received the full reference placebo infusion, 65 subjects received the full test placebo infusion, 63 subjects received the full crenezumab infusion, and 62 subjects received all three full infusions. On Day 1, subjects received a reference infusion of 4 mL placebo and a test infusion of placebo in which the volume increased volume across the cohorts (e.g., Cohort A: 4 mL, Cohort B: 8 mL, Cohort C: 12 mL, Cohort D: 16 mL, Cohort E: 20 mL, Cohort F: 24 mL, Cohort G: 32 mL and Cohort H: 40 mL). On Day 2, a single SC infusion of crenezumab was administered at equal volume to the test placebo infusion on Day 1 (e.g., 4-40 mL corresponding to 600-7200 mg of crenezumab). A formulation of 150 mg/mL crenezumab was used in Cohorts A-D and a 180 mg/mL formulation of crenezumab was used in Cohorts E-H. The crenezumab formulation contained succinic acid, L-arginine, and polysorbate 20. Infusions were delivered at a flow rate of approximately 1 mL/min via a Harvard Apparatus PHD Ultra™ 4400 Syringe Pump (Harvard Apparatus, Holliston, MA, USA) (e.g., 4-20 mL infusions) or a Canè Crono S-PID 50 Syringe Pump (Canè S.p.A. Medical Technology, Rivoli, Italy) (e.g., 24-40 mL infusions) via SC catheter (e.g., 25-gauge soft cannula inserted to a depth of 6 mm).

### Pain Assessment and LISSA Reactions

Tolerability was monitored by a Local Infusion Site Symptom Assessment tool (LISSA), a 100 mm Visual Analogue Scale (VAS), area under the VAS pain-time curve from time 0 to 60 minutes (AUC_{VAS0-60 min}), and Verbal Descriptive Scale (VDS) measurements. LISSA was performed for each infusion to document presence and size of infusion site reactions at pre-specified timepoints. Anonymized photographs were taken along with the LISSA assessment with a ruler on the subject's abdomen to estimate the size of local infusion site erythema. For the 0 minute and 60-minute timepoints, the infusion site photographs were calibrated using the ruler to determine the appropriate pixels/cm scaling factor. The area of erythema around the infusion site was manually segmented to determine the area (e.g., cm²) using image analysis software (e.g., ImageJ V1.5.2).

Infusion site leakage was evaluated using pre-weighed absorbent swabs to capture fluid on the site after catheter removal. Infusion site back pressure assessments were recorded for each infusion via a single-use pressure sensor connected between the syringe and infusion set. The pressure monitoring device recorded pressure once per second during infusion.

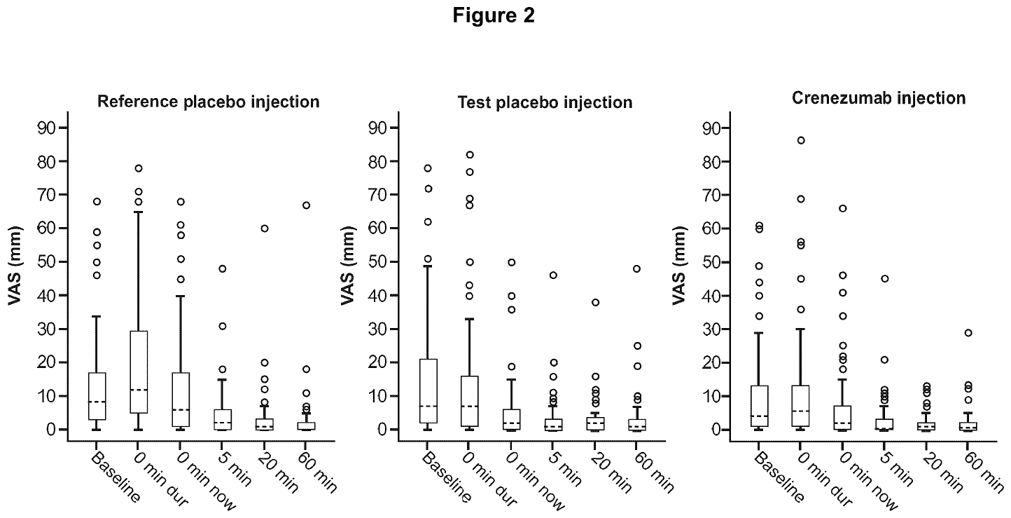

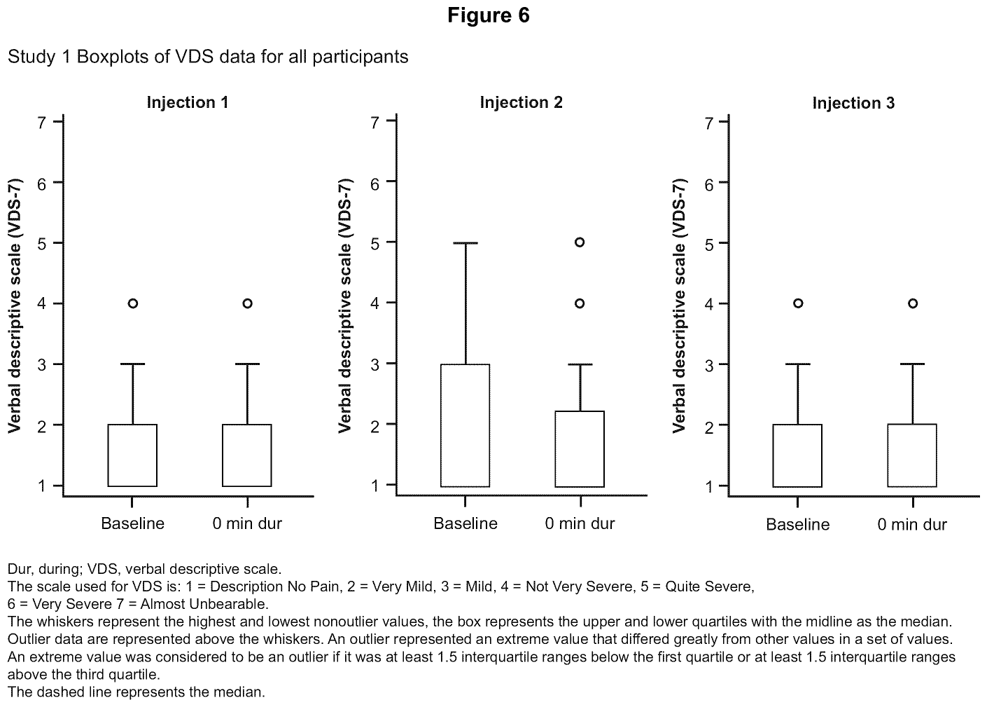

The highest mean VAS scores reported during the infusion for crenezumab, test placebo, and reference placebo administration were 18 mm (Cohort H), 28 mm (Cohort B), and 29 mm (Cohort C), respectively (FIG. 2). Mean VAS scores returned towards baseline after the end of the infusion. The highest mean VAS pain scores after the infusion were 25 mm at 0 minutes, 12 mm at 5 minutes, 10 mm at 20 minutes, and 9 mm at 60 minutes (all Cohort B, reference placebo). AUC_{VAS0-60 min} values were highest for reference placebo in cohorts B, C, D, E, and F, for test placebo in cohorts A and G, and for crenezumab in cohort H. Therefore, there were no differences in pain scores between the reference and test placebo infusions, or the test placebo and active crenezumab infusions, indicating there was no volume or drug effect. VDS scores for pain were also consistent with VAS assessments (FIG. 6).

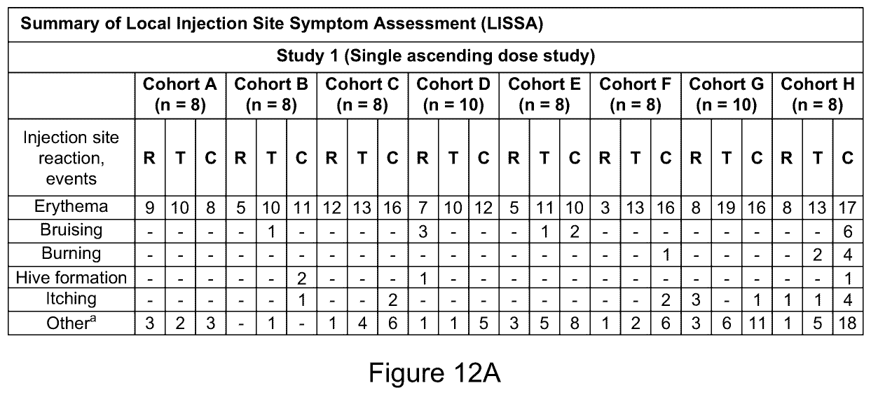

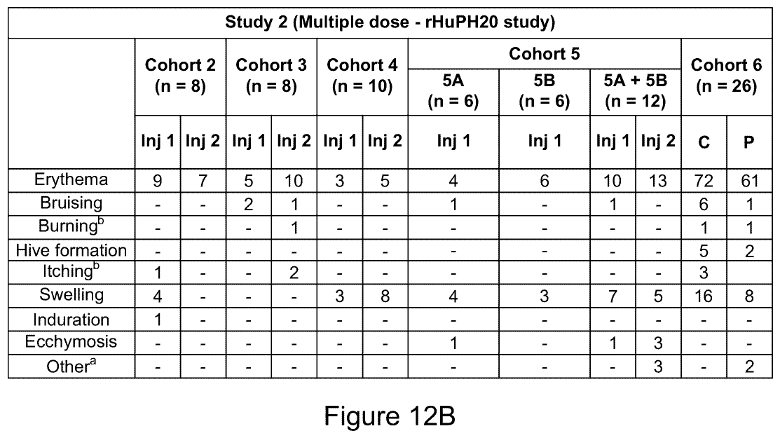

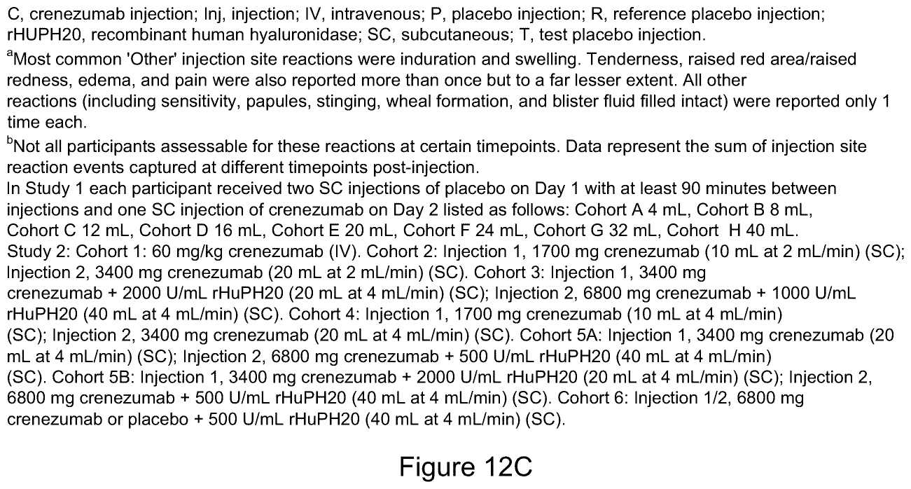

Infusion site erythema was the most commonly reported LISSA reaction (FIGS. 12A-12C). Erythema was reported in up to 100% subjects but did not appear to correlate with type of infusion received (e.g., crenezumab, test placebo, or reference placebo). Bruising, burning, and hive formation were reported in lower proportions (e.g., ≤37.5%). Most LISSA infusion site reactions were reported 60 minutes or 4 hours post-dose on Day 1 and resolved by the 72-hour assessment. There was no volume or drug effect when observing LISSA between reference placebo and test placebo, or between test placebo and active crenezumab infusions. Infusion site swelling or induration at any timepoint were reported more often in subjects aged <65 years (e.g., 57%; 24/42 subjects) compared with subjects aged ≥65 years (e.g., 38%; 10/26 subjects).

### Pharmacokinetics and Anti-Drug Antibodies Analysis

Serial blood samples for PK assessments were collected pre-dose and on days 3 (e.g., 24-hours post crenezumab infusion), 4, 5, 9, 16, 30, 44, 58 and 86. Blood samples were collected at baseline and at study completion or early termination for assessment of anti-drug antibodies (ADA) to crenezumab. Samples were analyzed using validated analytical methods. ADAs to crenezumab were analyzed by PPD Laboratories (Richmond, Virginia) using validated analytical procedures (LLOQ=50 ng/ml).

A quantitative assay designed to detect crenezumab in human serum was used for bioanalysis. Calibration standards, quality controls, and unknown samples were diluted to the assay minimum required dilution 1/50 in assay diluent. Diluted controls and samples were transferred to a pre-coated and pre-blocked enzyme-linked immunosorbent assay plate. During a 1-hour incubation, samples containing crenezumab were bound to immobilized aβ peptide. Unbound materials were removed with a wash step. Subsequently, horseradish peroxidase conjugated anti-human IgG was added for detection. Finally, tetramethylbenzidine peroxidase substrate was added to develop color. The substrate development was stopped after 15 to 30 minutes with 1 M phosphoric acid. The plate was read on a plate reader at 450 nm for detection absorbance, and at 630 nm for reference absorbance.

A qualitative assay designed to detect antibodies to MABT5102A in human serum was used for immunogenicity analysis. This assay used 2 conjugated reagents to capture antibodies directed against MABT5102A, biotin-conjugated MABT5102A (Biotin Conjugate Stock I) and digoxigenin-conjugated MABT5102A (digoxigenin Conjugate Stock I). These two conjugated reagents were co-incubated overnight at room temperature with the diluted controls and samples. The control/samples/biotin/digoxigenin solution was transferred to a NeutrAvidin™ protein-coated plate and incubated at room temperature. Following a wash step, a solution of mouse anti-digoxin antibody conjugated with horseradish peroxidase was added to the appropriate wells of the NeutrAvidin™-coated high-bind plate and incubated at room temperature. After a final wash step, a peroxidase substrate (tetramethylbenzidine) was added to the plate for color development and the reaction was stopped with 1 M phosphoric acid. The plate was read on a plate reader at 450 nm (detection) with a 630 nm reference filter.

Baseline prevalence of ADAs to crenezumab was calculated as the number of ADA-positive subjects relative to the number of evaluable subjects at baseline. The post-baseline incidences of ADAs to crenezumab antibodies were calculated as the number of ADA-positive subjects at post-baseline relative to the number of evaluable post-baseline subjects.

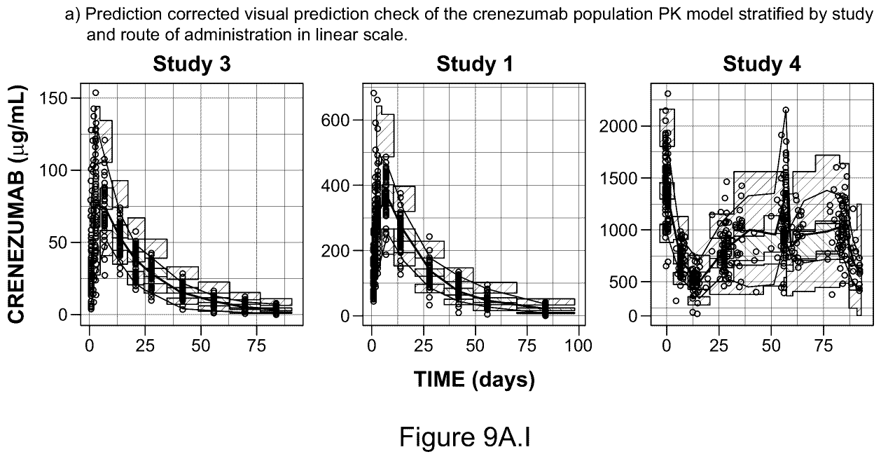

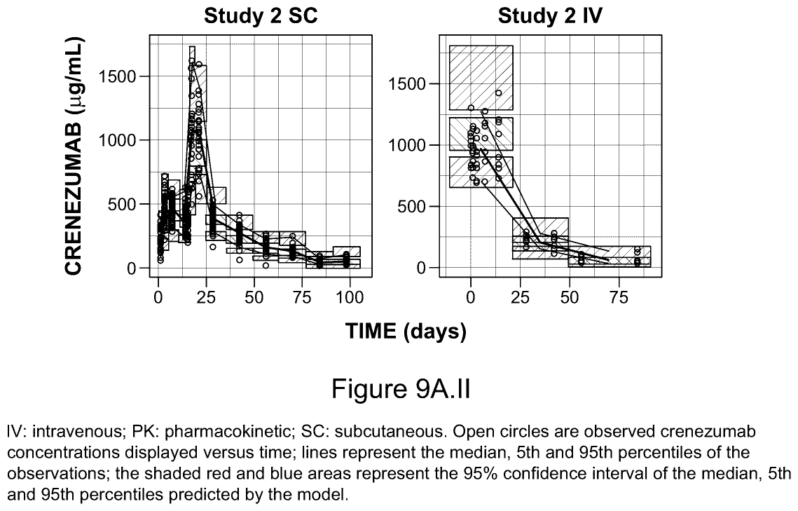

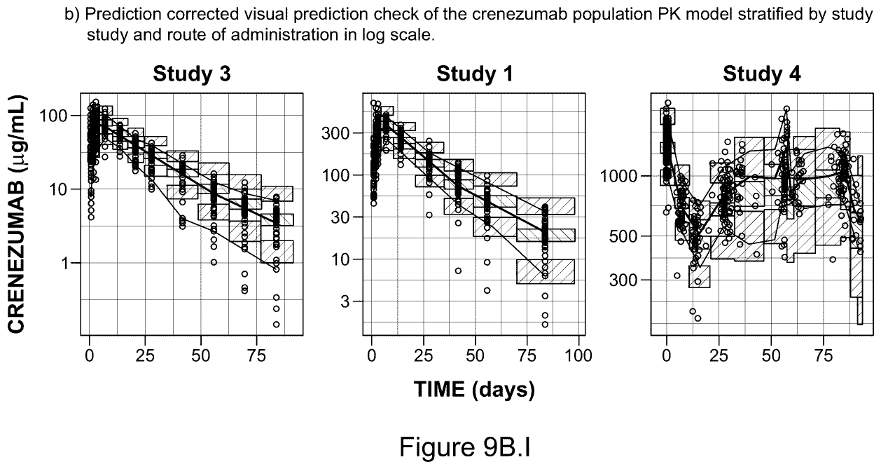

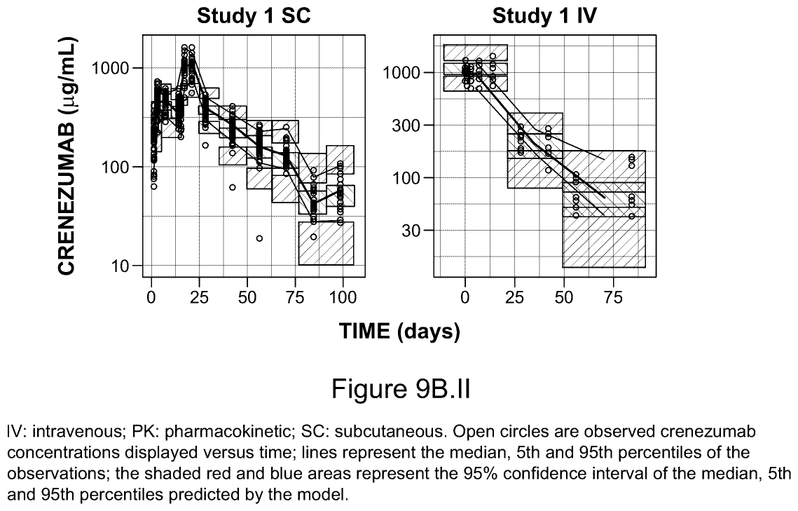

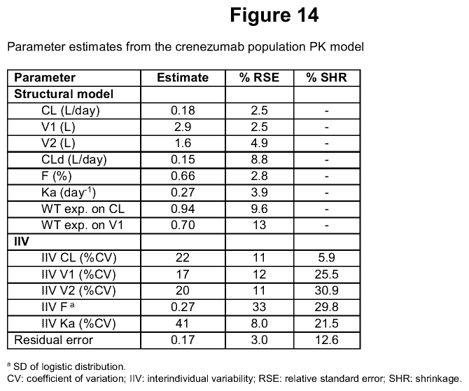

PK data from the Study 1, Study 2, Study 3 and Study 4 were used to develop the crenezumab population PK model. A two-compartment model with linear elimination and a depot compartment for SC absorption was developed to characterize crenezumab PK, using log transformed crenezumab concentrations. Model development was performed with nonlinear mixed effects modeling (NONMEM) v7.3 (Icon plc) and Pirana v2.9.9 (Certara, L. P.), with model evaluation and visualization performed in R v3.5.1. Body weight was a covariate on clearance and central volume (V1; FIG. 14), while other factors such as age and Alzheimer's disease status were not identified as covariates. Model evaluation was based on visual predictive check (VPC) of the final model presented in FIGS. 9A.I, 9A.II, 9B.I, and 9B.II. The VPC checks the ability of the model to simulate data similar to the data that was used for model development. Data was stratified based on study and route and was simulated 2000 times; 95% confidence interval was calculated for each of the percentiles in the VPCs.

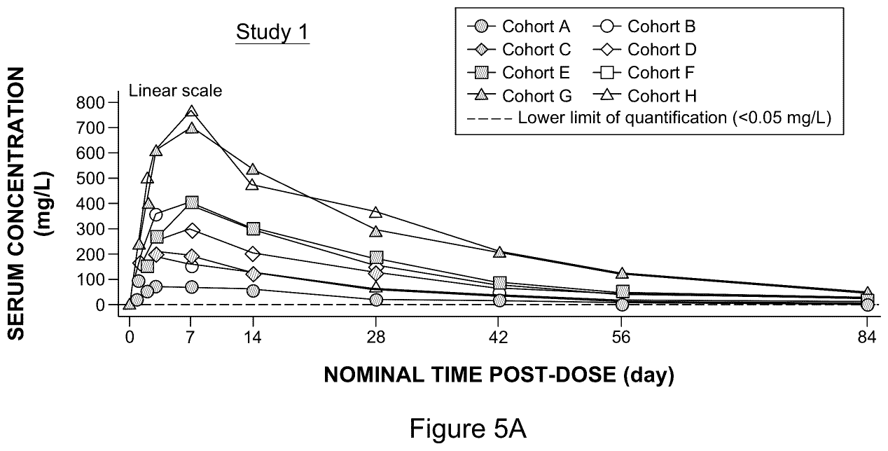

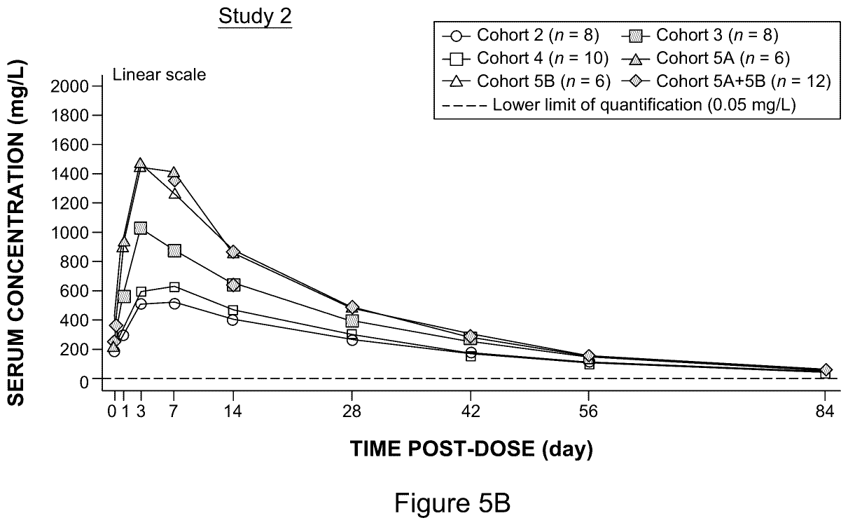

Mean serum concentration-time profiles were assessed following single ascending doses of SC crenezumab (e.g., 600-7200 mg) (FIGS. 5A and 5B). The Cₘₐₓ occurred at a median of 3-7 days (time to maximum observed concentration [Tₘₐₓ]) across all cohorts. After achieving Cₘₐₓ, serum concentrations of crenezumab declined with a mean terminal elimination half-life (t_{1/2}) of 17.0-24.9 days. Based on an ANOVA to assess dose proportionality, crenezumab Cₘₐₓ and AUC increased in an approximately dose-proportional manner over the dose range of 600-7200 mg.

The post-baseline incidence of ADAs to crenezumab was 2.9% (2 of 68 subjects). Both cases were considered to be treatment-emergent ADA to crenezumab with no apparent impact observed on PK or treatment-emergent adverse events (TEAE) findings.

### Treatment-Emergent Adverse Events

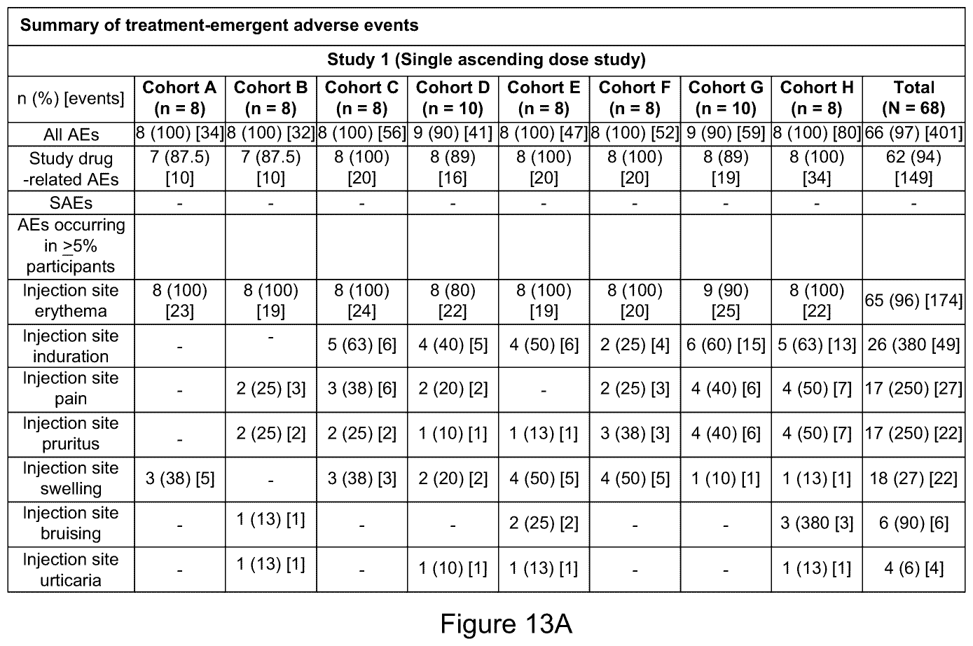

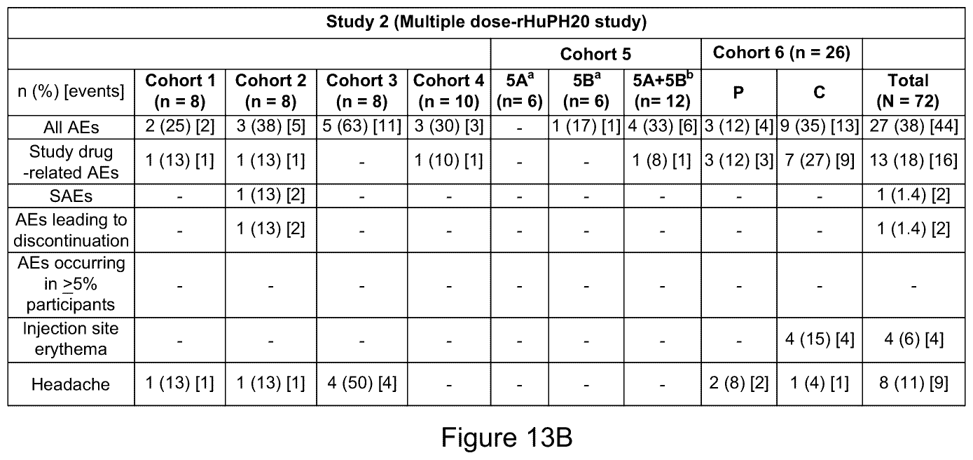

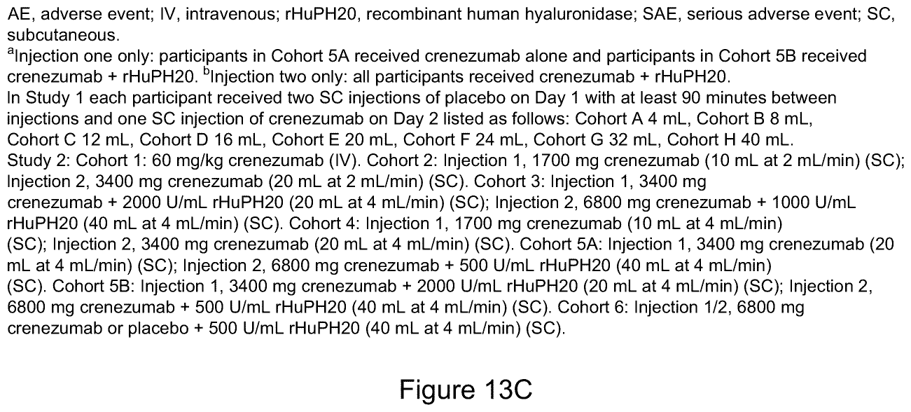

TEAEs were experienced by 63/66 (95.5%) subjects after to crenezumab (214 events), 63/67 (94.0%) after test placebo (108 events), and 51/68 (75.0%) after reference placebo (79 events) (FIGS. 13A-13C). Most TEAEs were Grade 1 in severity and infusion-related: 142/214 TEAEs after to crenezumab, 100/108 after test placebo, and 76/79 TEAEs after reference placebo. Six Grade ≥2 TEAEs were reported for to crenezumab in Cohort F (none were related to study drug in the opinion of the investigator). Five Grade 2 TEAEs were reported by two participants (pain in extremity, paraesthesia, peripheral swelling and thoracic vertebral fracture in one participant, and upper respiratory tract infection in the second participant). A Grade 3 macular hole was reported in the third participant. There were no serious or dose-limiting AEs in the study and no TEAEs led to study withdrawal. All TEAEs resolved by the end of the study. There was not an association between the type or incidence of TEAEs and the volume, drug, or dose administered.

### Example 2: Study of Safety and Tolerability of Different Combinations of Anti-Amyloid β Antibody, Infusion Volume, Flow Rate, and Concentration of Recombinant Human Hyaluronidase

A Phase I study (Study 2) was performed and recruited 72 healthy subjects (70 subjects completed the study) to assess the safety and tolerability of different combinations of crenezumab dose, infusion volume, flow rate, and concentration of recombinant human hyaluronidase (rHuPH20) administered subcutaneously. The pharmacokinetics (PK) of crenezumab after subcutaneous (SC) infusion with or without rHuPH20 was also assessed. The subjects were enrolled into one of six cohorts. Subjects were generally well-matched between cohorts in terms of age, weight, body mass index (BMI) and race; gender distribution varied (FIGS. 11A-11C). Cohorts 1-5 were open label and Cohort 6 was double-blinded (FIG. 1B). Subjects in Cohort 1 received a single 60 mg/kg IV infusion of crenezumab. Subjects in Cohorts 2-5 received two SC crenezumab infusions (e.g., 10-40 mL corresponding to 1700-6800 mg crenezumab across the cohorts) with rHuPH20 (e.g., Cohorts 3 and 5) or without rHuPH20 (e.g., Cohorts 2 and 4), on Days 1 and 15. Cohort 5 was divided into two groups: Group A received crenezumab alone for SC infusion on Day 1 and Group B received crenezumab plus rHuPH20 for SC infusion on Day 1. Both groups received crenezumab plus rHuPH20 for SC infusion on Day 15. Subjects in Cohort 6 received two SC infusions (≥90 but <180 minutes apart) on Day 1, one placebo and one crenezumab, both containing rHuPH20.

Population modelling of crenezumab PK for Cohorts 1-5 were used to determine the crenezumab dose for Cohort 6 (e.g., 6800 mg crenezumab) that was expected to provide similar crenezumab exposure (AUC) to an IV dose of 60 mg/kg. The sequence of infusions was randomized (e.g., block size of 4) and double-blinded. In Cohorts 2-6, SC infusion locations were randomized to different quadrants of the abdomen and no subjects received two infusions in the same quadrant. All infusions were delivered via SC catheter (e.g., 25-gauge soft cannula inserted to a depth of 6 mm) using the Harvard Apparatus PHD Ultra™ 4400 Syringe Pump.

### Pain Assessment and LISSA Reactions

Tolerability was monitored by a Local Infusion Site Symptom Assessment tool (LISSA; see supplementary information), a 100 mm Visual Analogue Scale (VAS), area under the VAS pain-time curve from time 0 to 60 minutes (AUC_{VAS0-60 min}), and Verbal Descriptive Scale (VDS) measurements. LISSA was performed for each infusion to document presence and size of infusion site reactions at pre-specified timepoints. Anonymized photographs were taken along with the LISSA assessment with a ruler on the subject's abdomen to estimate the size of local infusion site erythema. For the 0 minute and 60-minute timepoints, the infusion site photographs were calibrated using the ruler to determine the appropriate pixels/cm scaling factor. The area of erythema around the infusion site was manually segmented to determine the area (e.g., cm²) using image analysis software (e.g., ImageJ V1.5.2).

Infusion site leakage was evaluated using pre-weighed absorbent swabs to capture fluid on the site after catheter removal. Infusion site back pressure assessments were recorded for each infusion via a single-use pressure sensor connected between the syringe and infusion set. The pressure monitoring device recorded pressure once per second during infusion.

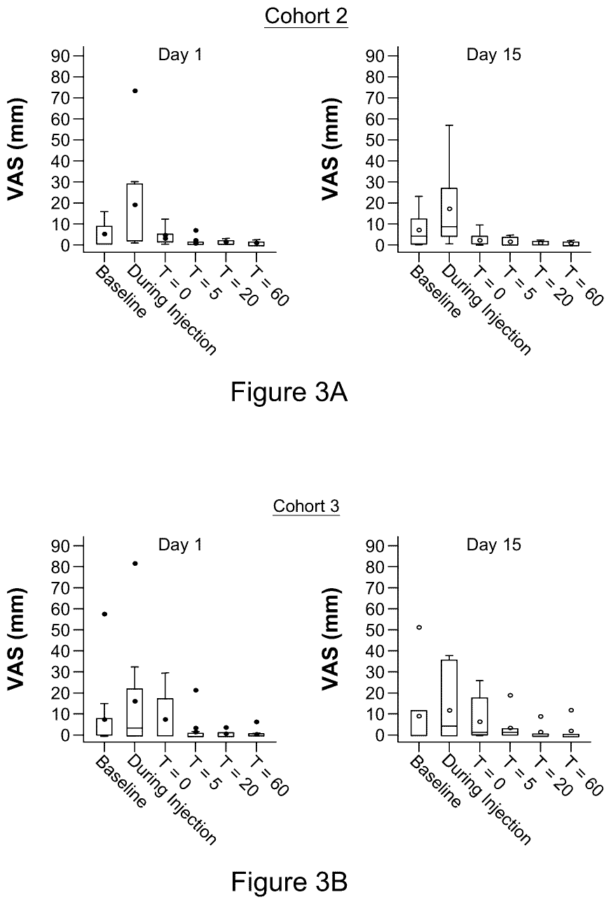

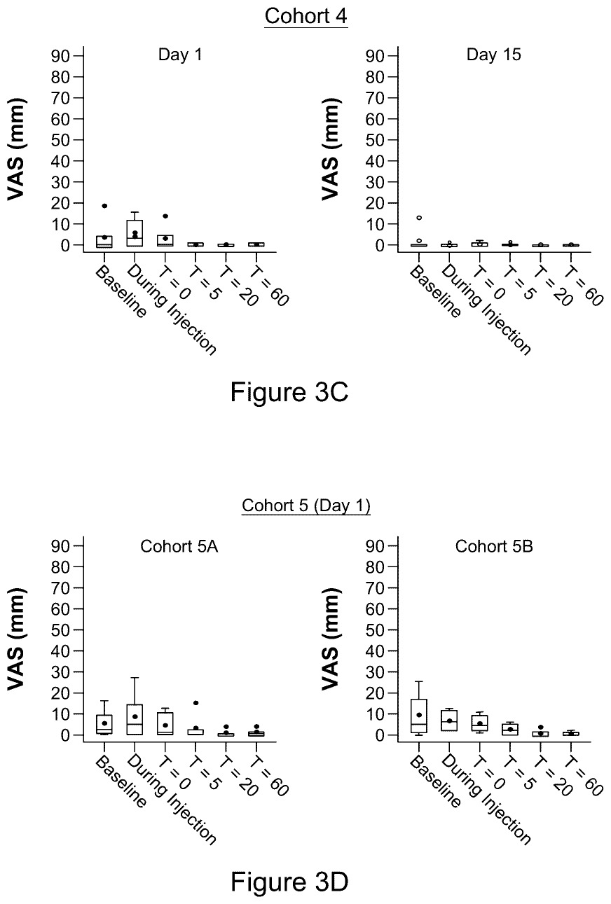

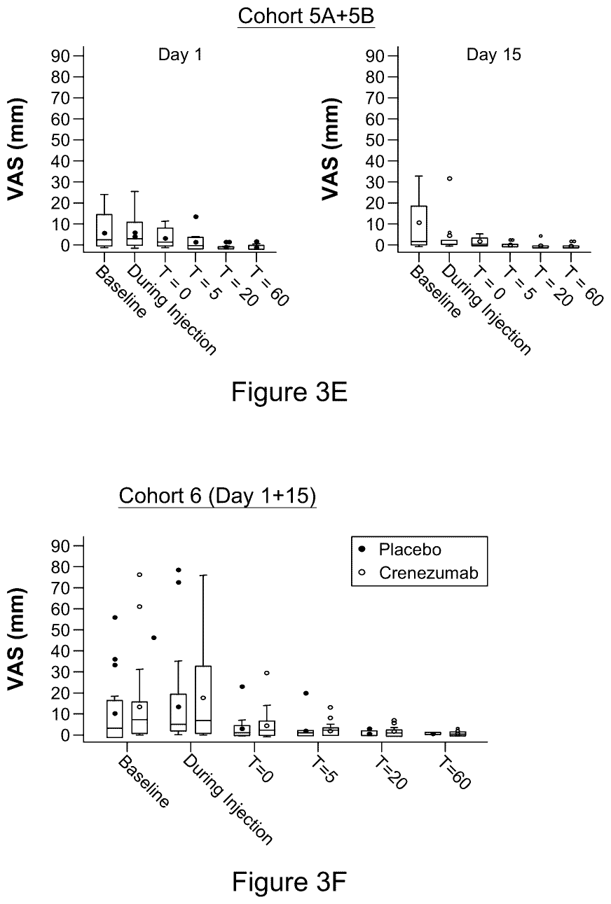

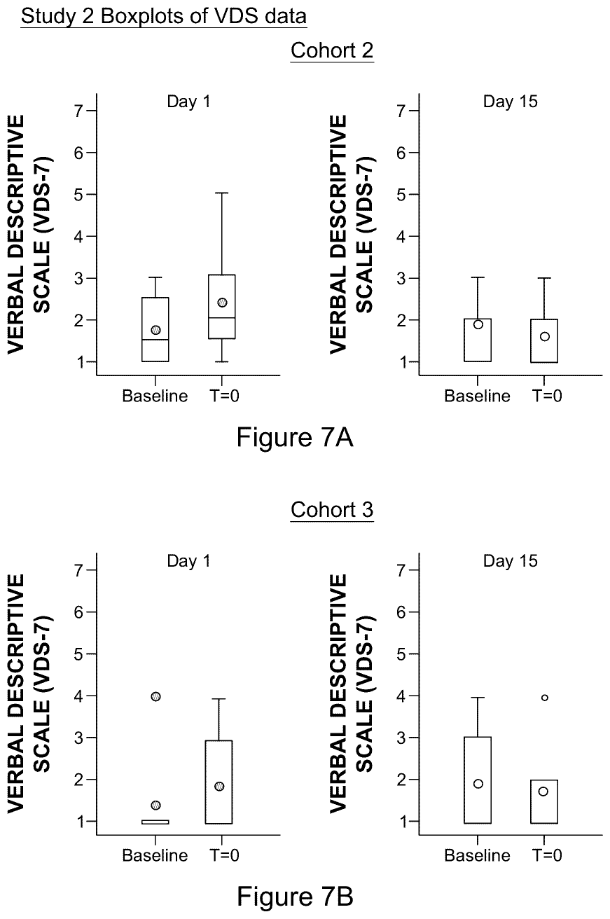

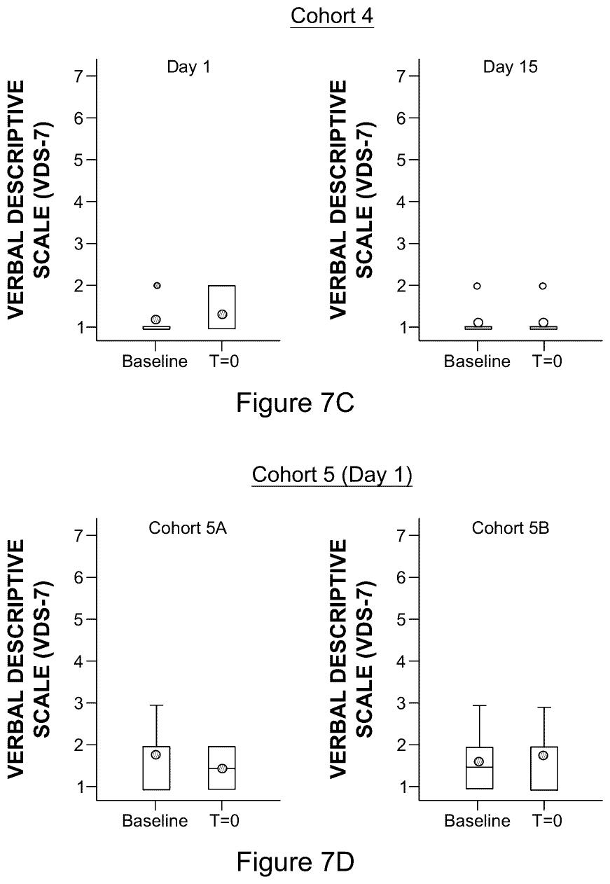

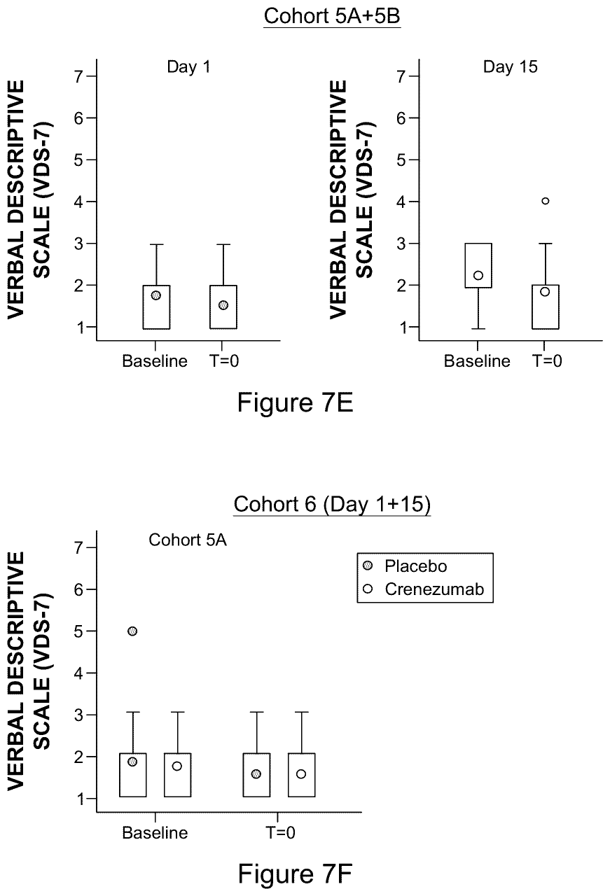

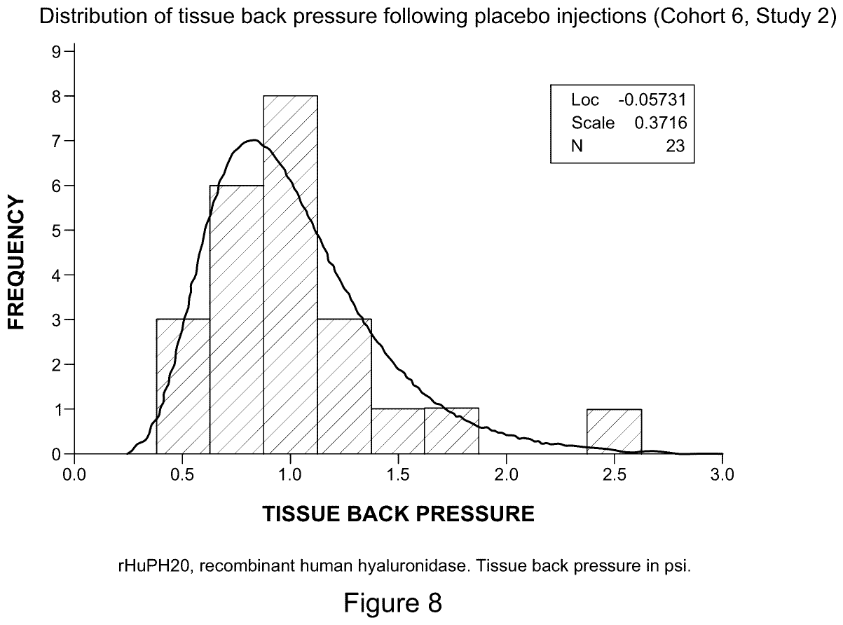

There were no differences observed between the baseline and post-dose VAS assessments. Subjects in Cohort 6 reported similar pain scores after receiving 40 mL crenezumab and rHuPH20, and after receiving 40 mL placebo and rHuPH20 (FIG. 3F). Therefore, there were no differences in mean VAS responses across treatments, suggesting that infusion flow rate, infusion volume, crenezumab dose in the presence or absence of rHuPH20, rHuPH20 concentration, and presence of crenezumab or placebo (e.g., Cohort 6) did not affect pain associated with the infusion. VDS scores for pain were consistent with VAS assessments (FIGS. 6 and 7A-7F).

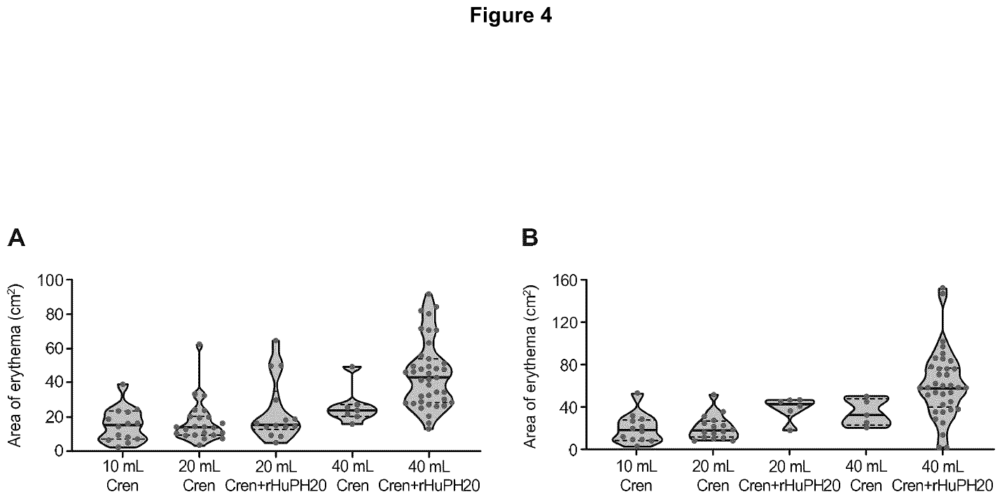

Infusion site erythema was a reported LISSA reaction (FIGS. 12A-12C). Erythema was reported in up to 92.3% of subjects within each cohort. The highest incidence was in Cohort 6 at 60 minutes post-infusion (e.g., 92.3% [24/26] after crenezumab plus rHuPH20 vs. 84.6% [22/26] after placebo plus rHuPH20). Incidence was similar regardless of crenezumab dose, infusion volume or amount of rHuPH20 administered. An analysis of infusion site photographs to determine the area of erythema was also performed (FIG. 4). The area of erythema at the 0-minute post-infusion timepoint appeared to be similar in subjects receiving 10 mL crenezumab, 20 mL crenezumab without rHuPH20, or 20 mL crenezumab with rHuPH20. At a volume of 40 mL, the area of erythema appeared larger with rHuPH20 co-administration than without, with both groups receiving 40 mL infusions tending to display larger areas of erythema than those given lower infusion volumes. At the 60-minute post-infusion timepoint, the area of erythema appeared similar in subjects receiving 10 mL crenezumab or 20 mL crenezumab without rHuPH20, with a trend towards larger areas of erythema in subjects receiving 20 mL crenezumab with rHuPH20. At a volume of 40 mL, the trends appeared to be similar to the 0-minute post-infusion timepoint, with larger areas of erythema in subjects receiving crenezumab with rHuPH20 co-administration than in subjects receiving crenezumab alone.

Infusion site swelling was another reported LISSA reaction (FIGS. 12A-12C). In Cohorts 2-5, swelling was more common after infusions not containing rHuPH20 compared with infusions that contained rHuPH20. In Cohort 6, incidence of infusion site erythema was not correlated with receipt of placebo or crenezumab. The incidence of swelling after crenezumab was approximately double that after placebo at the 0-minute (e.g., 8/26 [30.1%] vs. 4/26 [15.4%]) and 60-minute timepoints (e.g., 6/26 [23.1%] vs. 3/26 [11.5%]). Across Cohorts 2-6, infusion site swelling or induration at any timepoint were more commonly reported in subjects aged <65 years (e.g., 57%; 16/28 subjects) compared with subjects aged ≥65 years (e.g., 14%; 5/36 subjects).

### Pharmacokinetics

Serial blood samples were collected pre-dose and at 24-, 72-, 168-, and 336-hours post SC infusion of crenezumab, with or without rHuPH20, in all cohorts. Additional samples were collected on Days 29, 43, 57 and at study end (Day 85 for Cohorts 1 and 6, and Day 99 for all other cohorts). Cohort 1 also had a sample taken 60-90 minutes following IV infusion. Blood samples were collected at baseline and at study completion or early termination for assessment of anti-drug antibodies (ADA) to crenezumab, and anti-rHuPH20 antibodies. Samples were analyzed using validated analytical methods. ADAs to crenezumab were analyzed by Syneos (Princeton, NJ, USA). Analysis for anti-rHuPH20 was performed by Icon (Whitesboro, NY, USA) and for neutralizing antibodies to rHuPH20 by MicroConstants (San Diego, CA, USA).

A quantitative assay designed to detect crenezumab in human serum was used for bioanalysis. Calibration standards, quality controls, and unknown samples were diluted to the assay minimum required dilution 1/50 in assay diluent. Diluted controls and samples were transferred to a pre-coated and pre-blocked enzyme-linked immunosorbent assay plate. During a 1-hour incubation, samples containing crenezumab were bound to immobilized aβ peptide. Unbound materials were removed with a wash step. Subsequently, horseradish peroxidase conjugated anti-human IgG was added for detection. Finally, tetramethylbenzidine peroxidase substrate was added to develop color. The substrate development was stopped after 15 to 30 minutes with 1 M phosphoric acid. The plate was read on a plate reader at 450 nm for detection absorbance, and at 630 nm for reference absorbance.

A qualitative assay designed to detect antibodies to MABT5102A in human serum was used for immunogenicity analysis. This assay used 2 conjugated reagents to capture antibodies directed against MABT5102A, biotin-conjugated MABT5102A (Biotin Conjugate Stock I) and digoxigenin-conjugated MABT5102A (digoxigenin Conjugate Stock I). These two conjugated reagents were co-incubated overnight at room temperature with the diluted controls and samples. The control/samples/biotin/digoxigenin solution was transferred to a NeutrAvidin™ protein-coated plate and incubated at room temperature. Following a wash step, a solution of mouse anti-digoxin antibody conjugated with horseradish peroxidase was added to the appropriate wells of the NeutrAvidin™-coated high-bind plate and incubated at room temperature. After a final wash step, a peroxidase substrate (tetramethylbenzidine) was added to the plate for color development and the reaction was stopped with 1 M phosphoric acid. The plate was read on a plate reader at 450 nm (detection) with a 630 nm reference filter.

Baseline prevalence of ADAs to crenezumab and anti-rHuPH20 antibodies were calculated as the number of ADA-positive subjects relative to the number of evaluable subjects at baseline. The post-baseline incidences of ADAs to crenezumab and anti-rHuPH20 antibodies were calculated as the number of ADA-positive subjects at post-baseline relative to the number of evaluable post-baseline subjects.

PK data from Study 1, Study 2, Study 3 and Study 4 were used to develop the crenezumab population PK model. A two-compartment model with linear elimination and a depot compartment for SC absorption was developed to characterize crenezumab PK, using log transformed crenezumab concentrations. Model development was performed with nonlinear mixed effects modeling (NONMEM) v7.3 (Icon plc) and Pirana v2.9.9 (Certara, L. P.), with model evaluation and visualization performed in R v3.5.1.

After IV administration in Cohort 1, geometric mean Cₘₐₓ (e.g., 1370 mg/L) of crenezumab was achieved 0.08 days (˜2 hours) after administration and crenezumab concentration followed a biphasic distribution with a geometric mean terminal elimination t_{1/2} of 19.9 days. After SC administration of crenezumab in Cohorts 2-5, median Tₘₐₓ occurred at 3-7 days post-infusion. rHuPH20 had no effect on dose proportionality across cohorts or on t_{1/2}. Systemic exposure (Cₘₐₓ and AUC) was similar in subjects who received crenezumab alone (e.g., Cohort 5A) and crenezumab plus rHuPH20 (e.g., Cohort 5B). There was a trend, however, towards a shorter Tₘₐₓ with the rHuPH20-containing infusion (e.g., median 3 days in Cohort 5B vs 7 days in Cohort 5A). PK parameters were similar in Cohorts 2 and 4 indicating that the infusion flow rates tested (2 mL/min and 4 mL/min, respectively) did not affect crenezumab PK. After SC administration of 6800 mg crenezumab with 500 U/mL rHuPH20 in Cohort 6, median Tₘₐₓ was 3.1 days and arithmetic mean t_{1/2} was 20 days. Absolute bioavailability after SC administration in this cohort was 0.72 (calculated as DN AUC_{0-∞} (Cohort 6)/DN AUC_{0-∞} (Cohort 1).

A two-compartment pooled population PK model of crenezumab with linear elimination captured the observed PK data. PK data from 191 healthy subjects following single or multiple doses of crenezumab, and 62 patients with mild-to-moderate Alzheimer's disease following multiple doses of crenezumab were included in the pooled population PK analysis. Body weight was a covariate on clearance and central volume (V1; FIG. 14), while other factors such as age and Alzheimer's disease status were not identified as covariates. Model evaluation was based on visual predictive check (VPC) of the final model presented in FIGS. 9A.I, 9A.II, 9B.I, and 9B.II. The VPC checks the ability of the model to simulate data similar to the data that was used for model development. Data was stratified based on study and route and was simulated 2000 times; 95% confidence interval was calculated for each of the percentiles in the VPCs.

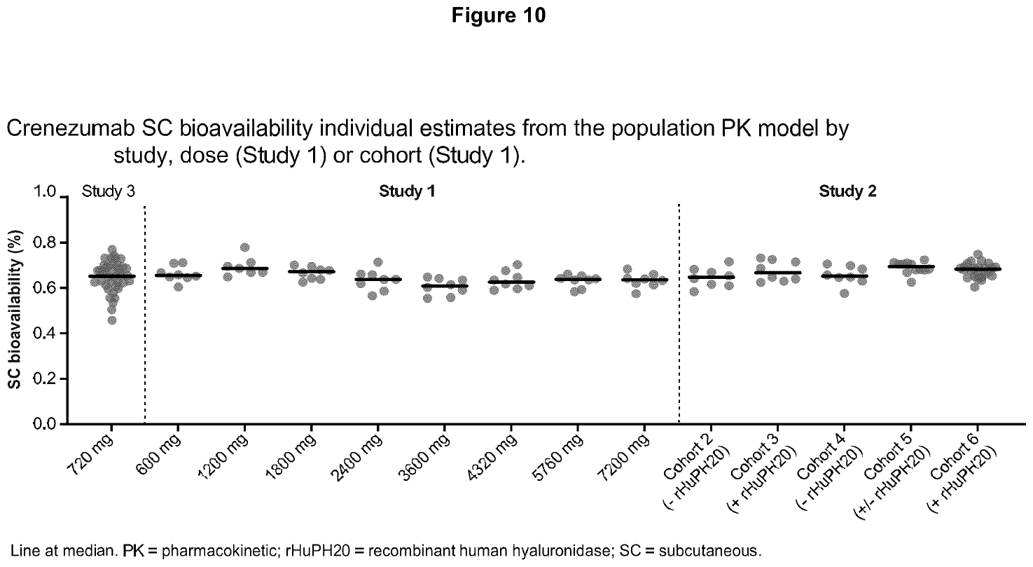

A logistic distribution was implemented for bioavailability (F) in order to constrain estimates between 0 and 1. Interindividual variability in CL, V1, V2, F, and Ka was estimated, with off-diagonal elements estimated between CL/V1/V2 (BLOCK3) and F/Ka (BLOCK2). An additive error model in the log domain was used to characterize residual error, reported as proportional residual error in the normal scale. Overall crenezumab SC bioavailability was estimated to be 0.66, which was similar when crenezumab was administered with and without rHuPH20 for both the study of Example 1 and Example 2 (FIG. 10).

Incidence of ADAs to crenezumab and anti-rHuPH20 antibodies was low following treatment (≤3%), with no apparent clinical impact on PK or safety in the studies of Example 1 and Example 2. No evidence of differential immunogenicity between SC crenezumab with or without rHuPH20 was observed, and the overall treatment emergent immunogenicity incidences were similar to those observed for IV crenezumab.

The baseline prevalence of ADAs to crenezumab was 2.8% (2 of 70). One subject in Cohort 2 and one subject in Cohort 4 had very low ADA titers (<1.70) prior to treatment and were ADA-negative post-baseline (treatment unaffected). The post-baseline incidence of ADA was 1.4% (1 of 70). One subject in Cohort 3 developed treatment-induced ADAs. Baseline prevalence of anti-rHuPH20 antibodies was 2.8% (e.g., One subject in Cohorts 3 and 6, unaffected by treatment) and the post-baseline incidence was also 2.8% (e.g., One subject in Cohort 5B and one subject in Cohort 6 had treatment-induced positive titers). Samples from all four subjects were negative for neutralizing antibodies to rHuPH20.

### Treatment-Emergent Adverse Events

37.5% (27/72) participants reported treatment-emergent adverse events (TEAEs) (44 events). Most TEAEs (86%) were mild (Grade 1) in severity, including the 16 considered to be crenezumab related (FIGS. 13A-13C). The most frequently reported TEAE (all causalities) was headache (nine TEAEs overall in 11.1% of participants). Grade 3 TEAEs included presyncope in one subject and increased blood creatine phosphokinase in a second participant. Two SAEs in one participant were attributed to a road traffic accident that resulted in a broken ankle and led to discontinuation of the participant from the study. Most were infusion-related and all were mild in severity. One subject in Cohort 6 experienced mild (Grade 1) oral hypoesthesia related to crenezumab that led to study drug withdrawal. This TEAE was resolved and the subject completed the study

### Example 3: Study Drug Preparation, Viscosity and Stability

In Study 1, a 150 mg/mL formulation of crenezumab was used in cohorts A-D and a 180 mg/mL formulation of crenezumab was used in cohorts E-H. In Study 2, all doses containing crenezumab were formulated at a concentration of 170 mg/mL (with or without rHuPH20). For doses of crenezumab without coformulation with rHuPH20, a 180 mg/mL formulation of crenezumab was diluted with placebo to reach a final concentration of 170 mg/mL. For doses of crenezumab with rHuPH20 at a concentration of 2000 U/mL, a manufactured formulation in vials was used directly without further compounding. Where a lower concentration of rHuPH20 was required, the vial formulation (170 mg/mL; 2000 U/mL) was diluted with appropriate volumes of crenezumab formulation (180 mg/mL) and placebo to achieve lower final rHuPH20 concentrations. For both studies, the crenezumab formulation contained succinic acid, L-arginine, and polysorbate 20; the placebo formulation contained the same excipients without the inclusion of crenezumab. At a crenezumab concentration of 170 mg/mL co-formulated with rHuPH20, mean viscosity at 5, 15 and 25° C. was 6.4, 9.0 and 13.7 centipoise, respectively.

Long-term stability in vials was confirmed independently at 5° C. and 25° ° C. “In-use” stability was also confirmed via incubation in polypropylene syringes for 24 h at 5° C. and 8 h at 25° C. and then subjected to simulated infusion.

1. A method for treating Alzheimer's disease in a subject, the method comprising subcutaneously administering to the subject a composition comprising a brain targeting antibody or antigen-binding fragment thereof, wherein the brain targeting antibody or antigen-binding fragment thereof is at a concentration of about 130 mg/mL to about 200 mg/mL.

2.-9. (canceled)

10. The method according to claim 1, wherein the concentration of the brain targeting antibody or antigen-binding fragment thereof is about 140 mg/mL to about 190 mg/mL.

11.-14. (canceled)

15. The method according to claim 1, wherein the brain targeting antibody or antigen-binding fragment thereof is administered at a dose between about 400 mg and about 7500 mg.

16.-27. (canceled)

28. The method according to claim 1, wherein the brain targeting antibody or antigen-binding fragment thereof is administered with an infusion volume of about 4 mL to about 60 mL.

29.-42. (canceled)

43. The method according to claim 1, further comprising administering to the subject a permeation enhancer.

44. The method according to claim 43, wherein the permeation enhancer is a recombinant human hyaluronidase.

45.-49. (canceled)

50. The method according to claim 44, wherein the concentration of the recombinant human hyaluronidase is about 500 U/mL to about 2000 U/mL.

51.-62. (canceled)

63. The method according to claim 1, wherein the brain targeting antibody or antigen-binding fragment thereof comprises: (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6.

64. The method according to claim 1, wherein the brain targeting antibody or antigen-binding fragment thereof comprises a VH domain comprising the amino acid sequence of SEQ ID NO: 7 and a VL domain comprising the amino acid sequence of SEQ ID NO: 8.

65. (canceled)

66. The method according to claim 1, wherein the brain targeting antibody comprises a heavy chain comprising the amino acid sequence of SEQ ID NO: 9 and a light chain comprising the amino acid sequence of SEQ ID NO: 10.

67. (canceled)

68. The method according to claim 1, wherein the brain targeting antibody is crenezumab.

69. (canceled)

70. A method for treating Alzheimer's disease in a subject, the method comprising subcutaneously administering to the subject a first dose and a second dose wherein:
(a) the first dose comprises about 1700 mg of a brain targeting antibody or antigen-binding fragment thereof, and is administered in an infusion volume of 10 mL, and with a flow rate of about 2 mL/min; and
(b) the second dose comprises about 3400 mg of a brain targeting antibody or antigen-binding fragment thereof, and is administered in an infusion volume of 20 mL, and with a flow rate of about 2 mL/min.

71. (canceled)

72. A method for treating Alzheimer's disease in a subject, the method comprising subcutaneously administering to the subject a first dose and a second dose wherein:
(a) the first dose comprises about 3400 mg of a brain targeting antibody or antigen-binding fragment thereof, and is administered in an infusion volume of 20 mL, and with a flow rate of about 4 mL/min; wherein said first dose is co-administered with recombinant human hyaluronidase at a dose of about 2000 U/mL; and
(b) the second dose comprises about 6800 mg of a brain targeting antibody or antigen-binding fragment thereof, and is administered in an infusion volume of 40 mL, and with a flow rate of about 4 mL/min; wherein said second dose is co-administered with recombinant human hyaluronidase at a dose of about 1000 U/mL.

73.-137. (canceled)
